(19) United States
(12) Patent Application Publication
(10) Pub. No.: US 2015/0329519 A1
(43) Pub. Date: Nov. 19, 2015

(54) Formulations Comprising Lipoyl Compounds

(71) Applicant: Ischemix, LLC, Maynard, MA (US)

(72) Inventors: Steven A. Kates, Needham, MA (US); Ralph Casale, Westford, MA (US); Alan S. Lader, Stoughton, MA (US); Alexander B. Baguisi, Grafton, MA (US); Reinier Beeuwkes, Concord, MA (US)

(21) Appl. No.: 14/710,074

(22) Filed: May 12, 2015

Related U.S. Application Data
(60) Provisional application No. 61/996,837, filed on May 14, 2014.

(51) Int. Cl. C07D 339/04; C07F 9/6553; C07K 5/065; C07K 5/062; C07K 5/072; C07K 5/078

(52) U.S. Cl. CPC C07D 339/04; C07K 5/06104; C07K 5/06147; C07K 5/06078; C07K 5/06026; C07F 9/655345

(57) ABSTRACT

Provided herein are aqueous pharmaceutical formulations comprising monomeric lipoyl compounds, such as compounds of Structural Formula I:

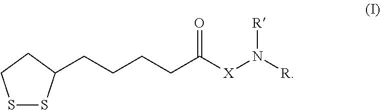

The formulations comprise a lipoyl compound comprising at least one acidic substituent; and an inorganic base in an amount sufficient to deprotonate each acidic substituent in the lipoyl compound. In certain embodiments, the formulations have a pH of from about 6.5 to about 8.0 and a tonicity of from about 250 mOsm to about 350 mOsm.

### RELATED APPLICATION(S)

This application claims the benefit of U.S. Provisional Application No. 61/996,837, filed on May 14, 2014. The entire teachings of the above application(s) are incorporated herein by reference.

### BACKGROUND OF THE INVENTION

Ischemic injuries are injuries resulting from restricted blood supply to an organ or tissue. Paradoxically, restoration of blood flow to affected tissues and organs following an ischemic episode can cause a secondary injury, ischemia-reperfusion injury. Ischemia-reperfusion injury often exacerbates the original ischemic injury, adding to the extent of organ or tissue damage.

Certain monomeric compounds containing a lipoyl moiety, such as those disclosed in International Publication No. WO2010/132657 and International Publication No. WO 2012/067947, have been shown to be efficacious for the treatment or prevention of ischemic and ischemia-reperfusion injuries. For example, in a human trial, α-N—[(R)-1,2-diothiolane-3-pentanoyl]-L-glutamyl-L-alanine [(R)Lip-EA-OH] showed statistically significant protection against myocardial damage associated with percutaneous coronary intervention (see Kates, S. A., et al. Bioorganic and Medicinal Chemistry 22 (2014) 505-512).

However, lipoyl compounds have a propensity to form impurities such as polymers upon exposure to light, a reaction proposed to proceed by photolytic opening of the dithiolane ring resulting in a diradical, followed by propagation through intermolecular disulfide bond formation (see Id. at 506). Potential degradation, including polymerization-induced degradation, of lipoyl compounds is, therefore, of concern in the development and formulation of therapeutic agents comprising lipoyl-containing compounds. Efforts to produce lipoyl compounds free of impurities, including contaminating polymeric impurities, have focused on crystallization and salt formation, neither of which are relevant to formulating lipoyl compounds for intravenous delivery.

Thus, there is a need for formulations comprising monomeric lipoyl compounds that are substantially free of their polymeric impurities as well as other impurities and can be administered safely to patients via an intravenous route of administration to treat or prevent ischemic injury or ischemia-reperfusion injuries.

### SUMMARY OF THE INVENTION

This invention relates to aqueous pharmaceutical formulations comprising monomeric lipoyl compounds (e.g., monomeric lipoyl compounds substantially free of impurities). The formulations comprise a lipoyl compound that is substituted with at least one acidic substituent; and an inorganic base in an amount sufficient to deprotonate each acidic substituent in the lipoyl compound. In certain embodiments, the formulations have a pH of from about 6.5 to about 8.0 and a tonicity of from about 250 mOsm to about 350 mOsm.

One embodiment of the invention relates to an aqueous pharmaceutical formulation, comprising a compound represented by Structural Formula I:

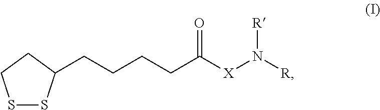

wherein the values and alternative values of variables X, R and R′ are as described and defined herein. The formulation further comprises an inorganic base in an amount sufficient to deprotonate each acidic substituent in the compound of Structural Formula I, and has a pH of from about 6.5 to about 8.0 and a tonicity of from about 250 mOsm to about 350 mOsm.

Another embodiment of the invention relates to an aqueous pharmaceutical formulation, comprising (i) from about 9 mg/mL to about 30 mg/mL of a compound represented by Structural Formula IIa:

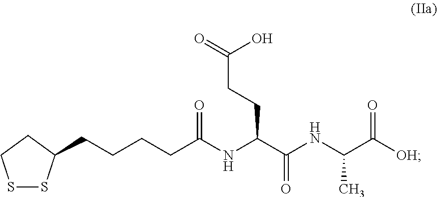 [SMILES: C[C@H](NC(=O)[C@H](CCC(=O)O)NC(=O)CCCC[C@@H]1CCSS1)C(=O)O]

(ii) from about 50 mg/mL to about 150 mg/mL sodium hydroxide; (iii) buffer; and (iv) a tonicity agent. The formulation has a pH of from about 6.8 to about 7.6 and a tonicity of from about 260 mOsm to about 320 mOsm. The compound of Structural Formula IIa (α-N—[(R)-1,2-diothiolane-3-pentanoyl]-L-glutamyl-L-alanine) is also referred to herein as (R)Lip-EA-OH.

Yet another embodiment of the invention relates to a process for preparing an aqueous pharmaceutical formulation. The process comprises providing a compound represented by Structural Formula I and providing an aqueous solution comprising an inorganic base in an amount sufficient to deprotonate each acidic substituent in the compound of Structural Formula I. The volume of the aqueous solution is equal to or greater than about 75% of the volume of the formulation. The compound of Structural Formula I is added to the aqueous solution, thereby forming a pharmaceutical solution, and the pharmaceutical solution is diluted to the volume of the formulation with a diluent to thereby prepare the aqueous pharmaceutical formulation.

In another embodiment, a process for preparing an aqueous pharmaceutical formulation having a pH of from about 6.5 to about 8.0 and a tonicity of from about 250 mOsm to about 350 mOsm comprises providing an aqueous solution comprising an amount of sodium hydroxide sufficient to form a formulation comprising from about 25 mg/mL to about 200 mg/mL sodium hydroxide, wherein the volume of the aqueous solution is equal to or greater than about 75% of the volume of the formulation. A compound represented by Structural Formula IIa is added to the aqueous solution in an amount sufficient to form a formulation comprising from about 5 mg/mL to about 50 mg/mL of the compound of Structural Formula IIa, thereby forming a pharmaceutical solution. The pharmaceutical solution is diluted to the volume of the formulation with a diluent, to prepare the aqueous pharmaceutical formulation having a pH of from about 6.5 to about 8.0 and a tonicity of from about 250 mOsm to about 350 mOsm.

Also provided herein are aqueous pharmaceutical formulations made according to the processes for preparing aqueous pharmaceutical formulations described herein.

The formulations described herein inhibit or eliminate the formation of undesired impurities, including polymeric impurities resulting from the polymerization of a monomeric lipoyl compound in the formulation. The formulations are stable for at least several months and, therefore, provide a means for delivering pure or substantially pure monomeric lipoyl compounds to a patient in an aqueous pharmaceutical formulation suitable for intravenous administration. The formulations can be administered safely to patients to treat or prevent ischemic and ischemia-reperfusion injuries.

### BRIEF DESCRIPTION OF THE DRAWINGS

The foregoing will be apparent from the following more particular description of example embodiments of the invention.

FIG. 1 is a copy of an HPLC chromatogram of the 30 mg/mL (R)Lip-EA-OH formulation resulting from the initial formulation protocol described in Example 2. (R)Lip-EA-OH was formulated by neutralizing solid (R)Lip-EA-OH with aqueous 1 N sodium bicarbonate and then diluting to volume with saline and water for injection. The chromatogram is enlarged to emphasize the products eluting in the baseline. The peak eluting at 12 minutes corresponds to (R)Lip-EA-OH. The broad peak eluting at about 15 minutes corresponds to the disulfide linked polymer of (R)Lip-EA-OH. Two other impurities are observed to be present in this lot at <0.4%, one of which is identified as R-lipoic acid.

FIG. 2 is a copy of an HPLC chromatogram of the 30 mg/mL (R)Lip-EA-OH formulation resulting from the revised formulation protocol described in Example 2. The formulation contains <0.1% polymer following formulation by adding (R)Lip-EA-OH to a solution of high pH and near final volume. The chromatogram is enlarged to emphasize the products eluting in the baseline. The peak eluting at 11.6 minutes corresponds to (R)Lip-EA-OH and the peak eluting at about 15.0 minutes corresponds to R-lipoic acid.

FIG. 3 is a bar graph of maximum CK-MB change from baseline as a function of treatment regimen (placebo or (R)Lip-EA-OH treatment at 0.8 mg/kg, 1.6 mg/kg or 2.4 mg/kg), and shows that patients in the (R)Lip-EA-OH treatment groups had less myocardial injury than patients in the placebo group, particularly at 2.4 mg/kg (R)Lip-EA-OH, following PCI.

FIG. 4 is a bar graph of troponin T change from baseline in the full analysis population (FAP) as a function of treatment regimen (placebo or (R)Lip-EA-OH treatment at 0.8 mg/kg, 1.6 mg/kg or 2.4 mg/kg), and shows that patients in the (R)Lip-EA-OH treatment groups had less myocardial injury than patients in the placebo group, particularly at 2.4 mg/kg (R)Lip-EA-OH, following PCI.

### DETAILED DESCRIPTION OF THE INVENTION

A description of example embodiments of the invention follows.

### Definitions

The disclosed compounds may exist in various stereoisomeric forms unless otherwise specified. “Stereoisomers” are compounds that differ only in their spatial arrangement. “Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms. “R” and “S” represent the configuration of substituents around one or more chiral carbon atoms.

“Racemate” or “racemic mixture,” as used herein, refers to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. When a single enantiomer is named or depicted by structure, the depicted or named enantiomer is present in an ee of at least or about 50%, at least or about 60%, at least or about 70%, at least or about 80%, at least or about 90%, at least or about 95%, at least or about 98%, at least or about 99% or at least or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D4 . . . ) represent the respective fractions of each diastereomer in a mixture, such that D1+(D2+D3+D4 . . . )=1. When a single diastereomer is named or depicted by structure, the depicted or named diastereomer is present in a de of at least or about 50%, at least or about 60%, at least or about 70%, at least or about 80%, at least or about 90%, at least or about 95%, at least or about 98%, at least or about 99% or at least or about 99.9%.

When a disclosed compound is named or depicted by structure without indicating the stereochemistry, and the compound has one chiral center, it is to be understood that the name or structure encompasses one enantiomer of the compound substantially separated from the corresponding optical isomer, a racemic mixture of the compound and mixtures enriched in one enantiomer relative to its corresponding optical isomer.

When a disclosed compound is named or depicted by structure without indicating the stereochemistry and has two or more chiral centers, it is to be understood that the name or structure encompasses a diastereomer substantially separated from other diastereomers, a pair of diastereomers substantially separated from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

“(R)-Lipoyl” refers to a compound containing a lipoyl moiety, wherein the stereocenter in the lipoyl moiety is in the (R) configuration. An (R)-lipoyl moiety is pictured below:

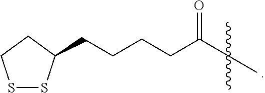 [SMILES: *C(=O)CCCC[C@@H]1CCSS1]

An example of an (R)-lipoyl compound is shown below:

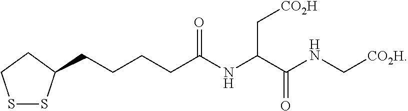 [SMILES: O=C(O)CNC(=O)C(CC(=O)O)NC(=O)CCCC[C@@H]1CCSS1]

“(S)-Lipoyl” refers to a compound containing a lipoyl moiety, wherein the stereocenter in the lipoyl moiety is in the (S) configuration. An (S)-lipoyl moiety is pictured below:

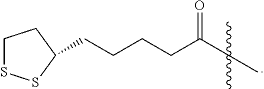 [SMILES: *C(=O)CCCC[C@H]1CCSS1 ; "."]

An example of an (S)-lipoyl compound is shown below:

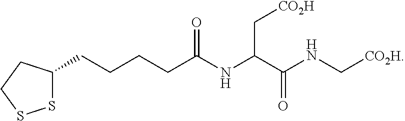 [SMILES: O=C(O)CNC(=O)C(CC(=O)O)NC(=O)CCCC[C@H]1CCSS1]

“Alkyl” means a saturated aliphatic branched or straight-chain monovalent hydrocarbon radical having the specified number of carbon atoms. Thus, “(C₁-C₆)alkyl” means a radical having from 1-6 carbon atoms in a linear or branched arrangement. “(C₁-C₆)alkyl” includes methyl, ethyl, propyl, i-propyl, butyl, i-butyl, t-butyl, sec-butyl, pentyl and hexyl. Typically, alkyl has 1 to 20, 1 to 15, 1 to 10, 1 to 5 or 1 to 3 carbon atoms.

One or more hydrogen atoms of an alkyl group can be replaced with a substituent group. Suitable substituent groups include hydroxy, thio, halo, halo(C₁-C₃)alkyl, (C₁-C₃)alkoxy and thio(C₁-C₃)alkyl. Preferred alkyl substituent groups include hydroxy and halo. An alkyl can also be substituted with one or more acidic substituents selected from the group consisting of —CO₂H, —SO₃H, —PO₃H₂, —OSO₃H, —OPO₃H₂, —B(OH)₂ and —NHOH.

The term “alkoxy” means —O-alkyl, where alkyl is as defined above.

The terms “halogen” and “halo” mean F, Cl, Br or I.

The term “thioalkyl” means —S-alkyl, where alkyl is as defined above.

The term “aryl” means a carbocyclic aromatic ring. “(C₆-C₁₄)aryl” includes phenyl, napthyl, indenyl, and anthracenyl. Typically, aryl has 6 to 20, 6 to 14, 6 to 10, 6 to 9, or 6 carbon atoms.

One or more hydrogen atoms of an aryl group can be replaced with a substituent group. Suitable substituent groups include hydroxy, thio, halo, (C₁-C₃)alkyl, halo(C₁-C₃)alkyl, (C₁-C₃)alkoxy and thio(C₁-C₃)alkyl. Preferred aryl substituent groups include hydroxy and halo. An aryl can also be substituted with one or more acidic substituents selected from the group consisting of —CO₂H, —SO₃H, —PO₃H₂, —OSO₃H, —OPO₃H₂, —B(OH)₂ and —NHOH.

As used herein, “substantially separated” or “substantially stereopure” means that the ee or de of the depicted or named compound is at least about 50%. For example, “substantially separated” or “substantially stereopure” can mean the ee or de of the depicted or named enantiomer is at least or about 50%, at least or about 60%, at least or about 70%, at least or about 80%, at least or about 90%, at least or about 95%, at least or about 98%, at least or about 99% or at least or about 99.9%. In one embodiment, substantially separated or substantially stereopure means that the ee or de of the depicted or named compound is at least or about 75%. In a specific embodiment, substantially separated means that the ee or de of the depicted or named compound is at least or about 90%. In a more specific embodiment, substantially separated means that the ee or de of the depicted or named compound is at least or about 95%. In yet a more specific embodiment, substantially separated means that the ee or de of the depicted or named compound is at least or about 99%. In another specific embodiment, substantially separated means that the ee or de of the depicted or named compound is at least or about 99.9%.

As used herein, the term “amino acid” means a molecule containing an amine group, a carboxylic acid group and a side chain which varies between different amino acids and includes both naturally-occurring amino acids and non-naturally-occurring amino acids. In one embodiment, “amino acid” is used to refer to naturally-occurring amino acids.

As used herein, the term “naturally-occurring amino acid” means a compound represented by the formula NH₂—CHR—COOH, wherein R is the side chain of a naturally-occurring amino acid such as an amino acid listed or named in the Table below. “Naturally-occurring amino acid” includes both the D- and L-configuration. When an amino acid is named or depicted by structure without indicating the stereochemistry and has at least one chiral center, it is to be understood that the name or structure encompasses a single enantiomer or diastereomer substantially separated from the other enantiomer or diastereomer, in which the one enantiomer or diastereomer is enriched relative to the other enantiomer or diastereomer(s), a racemic or diastereomeric mixture of the enantiomer or diastereomer(s) and mixtures enriched in one enantiomer or diastereomer relative to its corresponding optical isomer or other diastereomer(s). Preferred naturally occurring amino acids include aspartic acid, tyrosine, glutamic acid and alanine.

Table of Common Naturally Occurring Amino Acids

| | | Three | One |
|---|---|---|---|
| | | letter | letter |
| | Amino acid | code | code |
| Non-polar; | alanine | Ala | A |
| neutral at | isoleucine | Ile | I |
| pH 7.4 | leucine | Leu | L |
| | methionine | Met | M |
| | phenylalanine | Phe | F |
| | proline | Pro | P |
| | tryptophan | Trp | W |
| | valine | Val | V |
| Polar, | asparagine | Asn | N |
| uncharged | cysteine | Cys | C |
| at pH 7.0 | glycine | Gly | G |
| | glutamine | Gln | Q |
| | serine | Ser | S |
| | threonine | Thr | T |
| | tyrosine | Tyr | Y |
| Polar; | glutamic acid | Glu | E |
| charged at | arginine | Arg | R |
| pH 7 | aspartic acid | Asp | D |
| | histidine | His | H |
| | lysine | Lys | K |

“Non-natural amino acid” means an amino acid for which there is no nucleic acid codon. Examples of non-natural amino acids include natural α-amino acids with non-natural side chains; β-amino acids (e.g., β-alanine); γ-amino acids (e.g., γ-aminobutryric acid).

### Lipoyl Compounds

The present invention relates in one embodiment to aqueous pharmaceutical formulations comprising a compound represented by Structural Formula (I) and/or (Ia).

R is (C₁-C₁₈)alkyl, (C₆-C₁₈)aryl or (C₆-C₁₈)aryl(C₁-C₁₈)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆-C₁₈)aryl or (C₆-C₁₈)aryl(C₁-C₁₈)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

In one embodiment, R is (C₁-C₁₈)alkyl and is substituted with at least one acidic substituent selected from the group consisting of —CO₂H, —SO₃H, —PO₃H₂, —OSO₃H, —OPO₃H₂, —B(OH)₂ and —NHOH. In another embodiment, R is (C₁-C₃)alkyl and is substituted with at least one acidic substituent selected from the group consisting of —CO₂H, —SO₃H, —PO₃H₂, —OSO₃H, —OPO₃H₂, —B(OH)₂ and —NHOH. In a further embodiment, R is (C₃)alkyl and is substituted with at least one acidic substituent selected from the group consisting of —CO₂H, —SO₃H, —PO₃H₂, —OSO₃H, —OPO₃H₂, —B(OH)₂ and —NHOH. In a further embodiment, R is (C₂)alkyl and is substituted with at least one acidic substituent selected from the group consisting of —CO₂H, —SO₃H, —PO₃H₂, —OSO₃H, —OPO₃H₂, —B(OH)₂ and —NHOH. Alternatively, R is (C₁)alkyl and is substituted with at least one acidic substituent selected from the group consisting of —CO₂H, —SO₃H, —PO₃H₂, —OSO₃H, —OPO₃H₂, —B(OH)₂ and —NHOH.

In another embodiment, R is (C₆-C₁₈)aryl and is substituted with at least one acidic substituent selected from the group consisting of —CO₂H, —SO₃H, —PO₃H₂, —OSO₃H, —OPO₃H₂, —B(OH)₂ and —NHOH, and is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl. In a further embodiment, R is (C₆)aryl and is substituted with at least one acidic substituent selected from the group consisting of —CO₂H, —SO₃H, —PO₃H₂, —OSO₃H, —OPO₃H₂, —B(OH)₂ and —NHOH, and is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

In another embodiment, R is (C₆-C₁₈)aryl(C₁-C₁₈)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆-C₁₈)aryl(C₁-C₁₈)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl. In a further embodiment, R is (C₆)aryl(C₁-C₃)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆)aryl(C₁-C₃)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl. Alternatively, R is (C₆)aryl(C₁-C₂)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆)aryl(C₁-C₂)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

In another embodiment, R is (C₆)aryl(C₂)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆)aryl(C₂)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl. In a further embodiment, R is (C₆)aryl(C₁)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆)aryl(C₁)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

The at least one acidic substituent is selected from the group consisting of —CO₂H, —SO₃H, —PO₃H₂, —OSO₃H, —OPO₃H₂, —B(OH)₂ and —NHOH. In one embodiment, the at least one acidic substituent is selected from the group consisting of —CO₂H, —SO₃H, —PO₃H₂, —OSO₃H and —OPO₃H₂.

R is substituted with at least one acidic substituent selected from the group consisting of —CO₂H, —SO₃H, —PO₃H₂, —OSO₃H, —OPO₃H₂, —B(OH)₂ and —NHOH. In one embodiment, R is substituted with one, two or three acidic substituents. In a further embodiment, R is substituted with one or two acidic substituents.

Aryl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl. In one embodiment, aryl is further substituted with one, two or three substituents. In another embodiment, aryl is substituted with one substituent. Alternatively, aryl is unsubstituted. In a further embodiment, aryl is further substituted with one or more substituents selected from the group consisting of hydroxy or halo.

R′ is hydrogen or (C₁-C₁₈)alkyl, wherein said (C₁-C₁₈)alkyl is optionally substituted with one or more acidic substituents selected from the group consisting of —CO₂H, —SO₃H, —PO₃H₂, —OSO₃H, —OPO₃H₂, —B(OH)₂ and —NHOH. In one embodiment, R′ is hydrogen.

In one embodiment, R′ is (C₁-C₁₈)alkyl. In another embodiment, R′ is (C₁-C₃)alkyl. In a further embodiment, R′ is (C₃)alkyl. In a further embodiment, R′ is (C₂)alkyl. Alternatively, R′ is (C₁)alkyl.

R′ is substituted with at least one acidic substituent selected from the group consisting of —CO₂H, —SO₃H, —PO₃H₂, —OSO₃H, —OPO₃H₂, —B(OH)₂ and —NHOH. In one embodiment, R′ is substituted with one, two or three acidic substituents. In another embodiment, R′ is substituted with one or two acidic substituents. In a further embodiment, R′ is substituted with one acidic substituent. Alternatively, R′ is unsubstituted.

X is absent or an amino acid, wherein the amino acid is oriented to form an amide linkage with

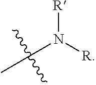

For example, the moiety in N-lipoyl-glutamylalanine is oriented as shown in the structural formula below:

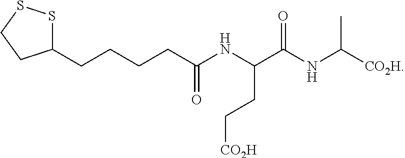 [SMILES: CC(NC(=O)C(CCC(=O)O)NC(=O)CCCCC1CCSS1)C(=O)O]

In one embodiment, X is absent. Alternatively, X is an amino acid. In a further embodiment, X is a naturally-occurring amino acid. In yet a further embodiment, X is aspartic acid, tyrosine, glutamic acid or alanine.

In a 1^{st} specific embodiment, the compound is represented by Structural Formula (I) and/or (Ia), wherein the values and alternative values for the variables are as described above.

In a first aspect of the 1^{st} specific embodiment of the present invention, the (R)-lipoyl stereoisomer of a compound represented by Structural Formulas (I) or (Ia) is substantially separated from the (S)-lipoyl stereoisomer(s). Values and alternative values for the remainder of the variables are as described above for Structural Formulas (I) or (Ia) or in the 1^{st} specific embodiment.

In a second aspect of the 1^{st} specific embodiment of the present invention, R′ is H. Values and alternative values for the remainder of the variables are as described above for Structural Formulas (I) or (Ia) or in the 1^{st} specific embodiment, or first aspect thereof.

In a third aspect of the 1^{st} specific embodiment of the present invention, R′ is H and X is a naturally-occurring amino acid. Values and alternative values for the remainder of the variables are as described above for Structural Formulas (I) or (Ia) or in the 1^{st} specific embodiment, or first or second aspect thereof.

In a fourth aspect of the 1^{st} specific embodiment of the present invention, R and R′ are each (C₁-C₃)alkyl substituted with one or two acidic substituents each independently selected from the group consisting of —CO₂H, —SO₃H, —PO₃H₂, —OSO₃H and —OPO₃H₂. Values and alternative values for the remainder of the variables are as described above for Structural Formulas (I) or (Ia) or in the 1^{st} specific embodiment, or first to third aspects thereof.

In a fifth aspect of the 1^{st} specific embodiment of the present invention, R′ is H and X is absent. Values and alternative values for the remainder of the variables are as described above for Structural Formulas (I) or (Ia) or in the 1^{st} specific embodiment, or first to fourth aspects thereof.

In a sixth aspect of the 1^{st} specific embodiment of the present invention, R is (C₁-C₃)alkyl substituted with one or two acidic substituents each independently selected from the group consisting of —CO₂H, —SO₃H, —PO₃H₂, —OSO₃H and —OPO₃H₂. Values and alternative values for the remainder of the variables are as described above for Structural Formulas (I) or (Ia) or in the 1^{st} specific embodiment, or first to fifth aspects thereof.

In a seventh aspect of the 1^{st} specific embodiment of the present invention, R is (C₆)aryl(C₁-C₃)alkyl substituted with one or two acidic substituents each independently selected from the group consisting of —CO₂H, —SO₃H, —PO₃H₂, —OSO₃H and —OPO₃H₂. Values and alternative values for the remainder of the variables are as described above for Structural Formulas (I) or (Ia) or in the 1^{st} specific embodiment, or first to sixth aspects thereof.

In an eighth aspect of the 1^{st} specific embodiment of the present invention, R is (C₂)alkyl substituted with one or two acidic substituents each independently selected from the group consisting of —CO₂H, —SO₃H, —PO₃H₂, —OSO₃H and —OPO₃H₂. Values and alternative values for the remainder of the variables are as described above for Structural Formulas (I) or (Ia) or in the 1^{st} specific embodiment, or first to seventh aspects thereof.

In a ninth aspect of the 1^{st} specific embodiment of the present invention, R is (C₆)aryl substituted with one acidic substituent selected from the group consisting of —CO₂H, —SO₃H, —PO₃H₂, —OSO₃H and —OPO₃H₂. Values and alternative values for the remainder of the variables are as described above for Structural Formulas (I) or (Ia) or in the 1^{st} specific embodiment, or first to eighth aspects thereof.

In a tenth aspect of the first specific embodiment, the compound is represented by Structural Formula (I), wherein the values and alternative values are as described above for Structural Formulas (I) or (Ia) or in the 1^{st} specific embodiment, or first to ninth aspects thereof.

In an eleventh aspect of the 1^{st} specific embodiment, the compound is represented by Structural Formula (Ia), wherein the values and alternative values are as described above for Structural Formulas (I) or (Ia) or in the 1^{st} specific embodiment, or first to tenth aspects thereof.

In a 2^{nd} specific embodiment, the compound is represented by any one of the structural formulas in Table A.

TABLE A

| Cpd | | Cpd | |
|---|---|---|---|
| No. | Structural Formula | No. | Structural Formula |
| A | 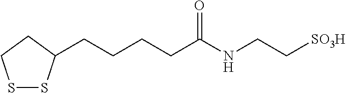 [SMILES: O=C(CCCCC1CCSS1)NCCS(=O)(=O)O] | A′ | 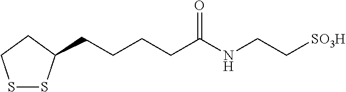 [SMILES: O=C(CCCC[C@@H]1CCSS1)NCCS(=O)(=O)O] |
| B | 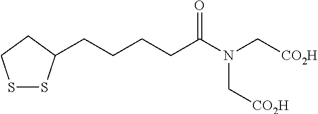 [SMILES: O=C(O)CN(CC(=O)O)C(=O)CCCCC1CCSS1] | B′ | 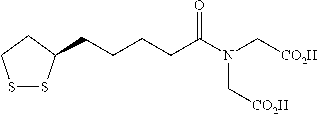 [SMILES: O=C(O)CN(CC(=O)O)C(=O)CCCC[C@@H]1CCSS1] |
| C | 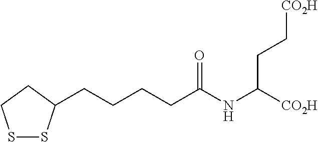 [SMILES: O=C(O)CCC(NC(=O)CCCCC1CCSS1)C(=O)O] | C′ | 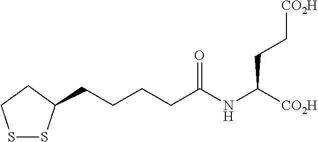 [SMILES: O=C(O)CC[C@H](NC(=O)CCCC[C@@H]1CCSS1)C(=O)O] |
| D | 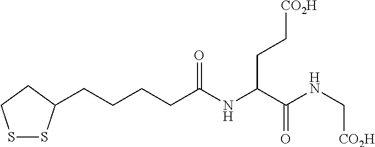 [SMILES: O=C(O)CCC(NC(=O)CCCCC1CCSS1)C(=O)NCC(=O)O] | D′ | 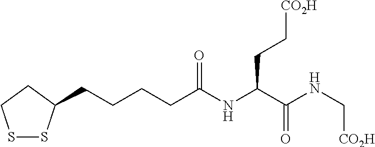 [SMILES: O=C(O)CC[C@H](NC(=O)CCCC[C@@H]1CCSS1)C(=O)NCC(=O)O] |
| E | 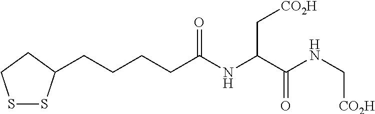 [SMILES: O=C(O)CNC(=O)C(CC(=O)O)NC(=O)CCCCC1CCSS1] | E′ | 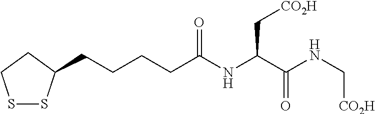 [SMILES: O=C(O)CNC(=O)[C@H](CC(=O)O)NC(=O)CCCC[C@@H]1CCSS1] |
| F | 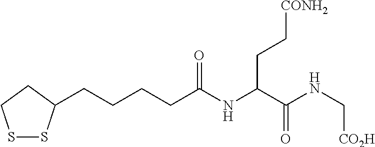 [SMILES: NC(=O)CCC(NC(=O)CCCCC1CCSS1)C(=O)NCC(=O)O] | F′ | 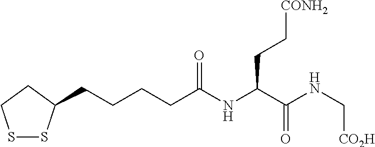 [SMILES: NC(=O)CC[C@H](NC(=O)CCCC[C@@H]1CCSS1)C(=O)NCC(=O)O] |
| G | 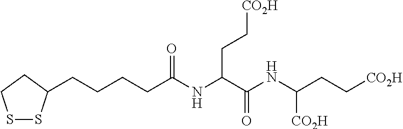 [SMILES: O=C(O)CCC(NC(=O)C(CCC(=O)O)NC(=O)CCCCC1CCSS1)C(=O)O] | G′ | 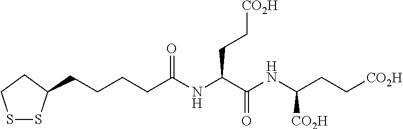 [SMILES: O=C(O)CC[C@H](NC(=O)[C@H](CCC(=O)O)NC(=O)CCCC[C@@H]1CCSS1)C(=O)O] |
| H | 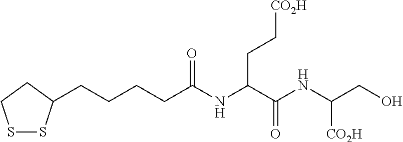 [SMILES: O=C(O)CCC(NC(=O)CCCCC1CCSS1)C(=O)NC(CO)C(=O)O] | H′ | 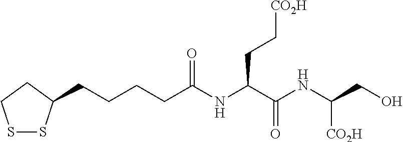 [SMILES: O=C(O)CC[C@H](NC(=O)CCCC[C@@H]1CCSS1)C(=O)N[C@@H](CO)C(=O)O] |
| I | 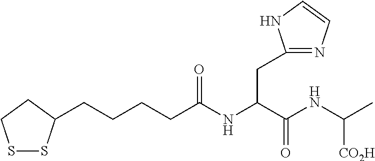 [SMILES: CC(NC(=O)C(Cc1ncc[nH]1)NC(=O)CCCCC1CCSS1)C(=O)O] | I′ | 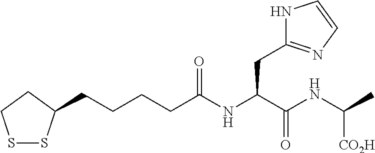 [SMILES: C[C@H](NC(=O)[C@H](Cc1ncc[nH]1)NC(=O)CCCC[C@@H]1CCSS1)C(=O)O] |
| J | 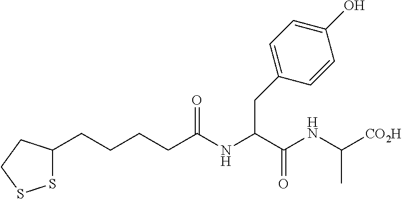 [SMILES: CC(NC(=O)C(Cc1ccc(O)cc1)NC(=O)CCCCC1CCSS1)C(=O)O] | J′ | 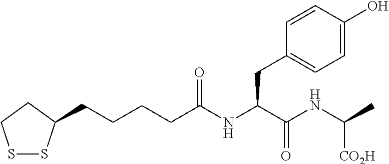 [SMILES: C[C@H](NC(=O)[C@H](Cc1ccc(O)cc1)NC(=O)CCCC[C@@H]1CCSS1)C(=O)O] |
| K | 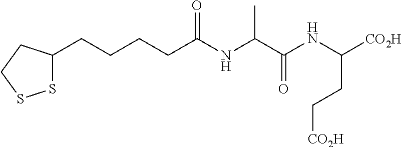 [SMILES: CC(NC(=O)CCCCC1CCSS1)C(=O)NC(CCC(=O)O)C(=O)O] | K′ | 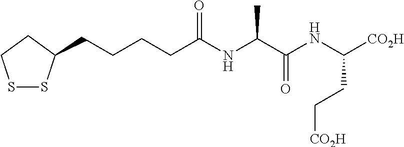 [SMILES: C[C@H](NC(=O)CCCC[C@@H]1CCSS1)C(=O)N[C@@H](CCC(=O)O)C(=O)O] |
| L | 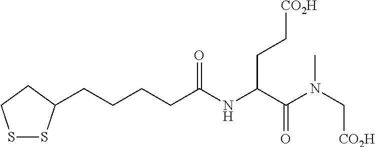 [SMILES: CN(CC(=O)O)C(=O)C(CCC(=O)O)NC(=O)CCCCC1CCSS1] | L′ | 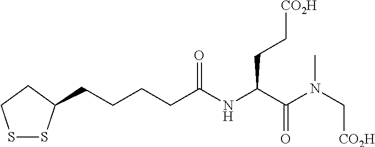 [SMILES: CN(CC(=O)O)C(=O)[C@H](CCC(=O)O)NC(=O)CCCC[C@@H]1CCSS1] |
| M | 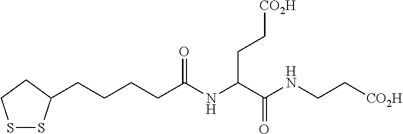 [SMILES: O=C(O)CCNC(=O)C(CCC(=O)O)NC(=O)CCCCC1CCSS1] | M′ | 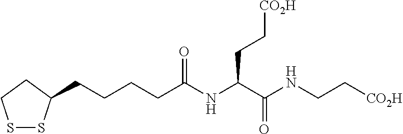 [SMILES: O=C(O)CCNC(=O)[C@H](CCC(=O)O)NC(=O)CCCC[C@@H]1CCSS1] |
| N | 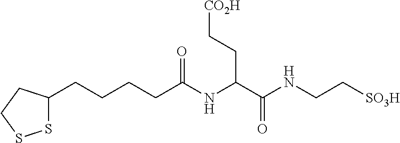 [SMILES: O=C(O)CCC(NC(=O)CCCCC1CCSS1)C(=O)NCCS(=O)(=O)O] | N′ | 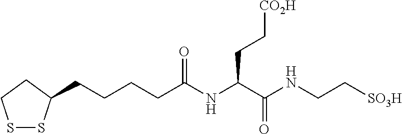 [SMILES: O=C(O)CC[C@H](NC(=O)CCCC[C@@H]1CCSS1)C(=O)NCCS(=O)(=O)O] |
| O | 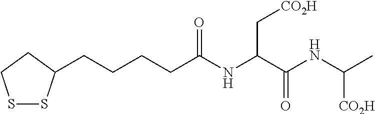 [SMILES: CC(NC(=O)C(CC(=O)O)NC(=O)CCCCC1CCSS1)C(=O)O] | O′ | 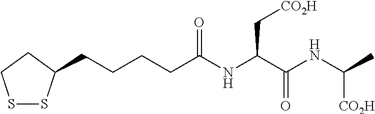 [SMILES: C[C@H](NC(=O)[C@H](CC(=O)O)NC(=O)CCCC[C@@H]1CCSS1)C(=O)O] |
| Q | 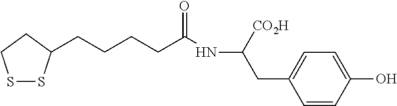 [SMILES: O=C(CCCCC1CCSS1)NC(Cc1ccc(O)cc1)C(=O)O] | Q′ | 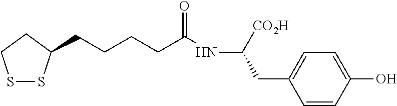 [SMILES: O=C(CCCC[C@@H]1CCSS1)N[C@@H](Cc1ccc(O)cc1)C(=O)O] |
| R | 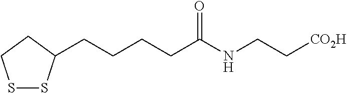 | R′ | 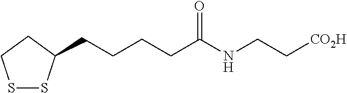 |
| S | 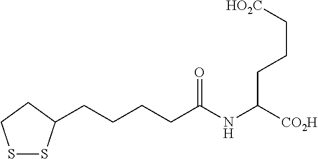 [SMILES: O=C(O)CCCC(NC(=O)CCCCC1CCSS1)C(=O)O] | S′ | 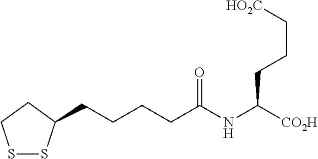 [SMILES: O=C(O)CCC[C@H](NC(=O)CCCC[C@@H]1CCSS1)C(=O)O] |
| T | 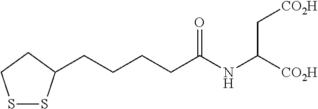 [SMILES: O=C(O)CC(NC(=O)CCCCC1CCSS1)C(=O)O] | T ′ | 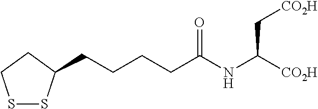 [SMILES: O=C(O)C[C@H](NC(=O)CCCC[C@@H]1CCSS1)C(=O)O] |
| U | 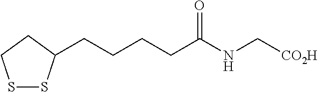 [SMILES: O=C(O)CNC(=O)CCCCC1CCSS1] | U′ | 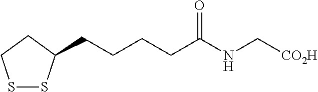 [SMILES: O=C(O)CNC(=O)CCCC[C@@H]1CCSS1] |
| V | 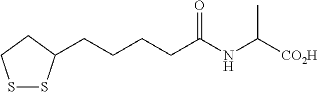 [SMILES: CC(NC(=O)CCCCC1CCSS1)C(=O)O] | V′ | 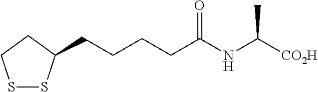 [SMILES: C[C@H](NC(=O)CCCC[C@@H]1CCSS1)C(=O)O] |
| W | 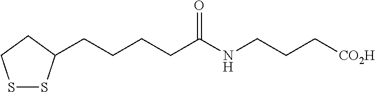 [SMILES: O=C(O)CCCNC(=O)CCCCC1CCSS1] | W′ | 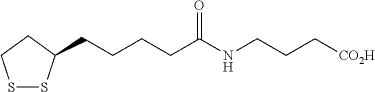 [SMILES: O=C(O)CCCNC(=O)CCCC[C@@H]1CCSS1] |
| X | 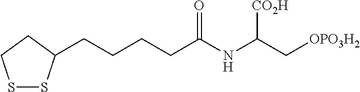 | X′ | 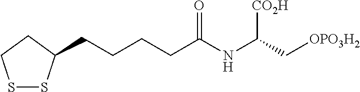 |
| Y | 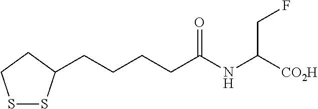 [SMILES: O=C(CCCCC1CCSS1)NC(CF)C(=O)O] | Y′ | 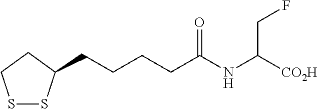 [SMILES: O=C(CCCC[C@@H]1CCSS1)NC(CF)C(=O)O] |
| Z | 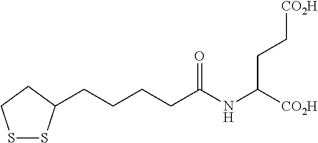 [SMILES: O=C(O)CCC(NC(=O)CCCCC1CCSS1)C(=O)O] | Z′ | 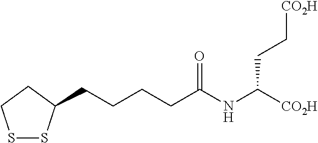 [SMILES: O=C(O)CC[C@@H](NC(=O)CCCC[C@@H]1CCSS1)C(=O)O] |
| AB | 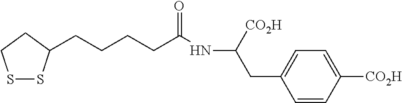 [SMILES: O=C(CCCCC1CCSS1)NC(Cc1ccc(C(=O)O)cc1)C(=O)O] | AB′ | 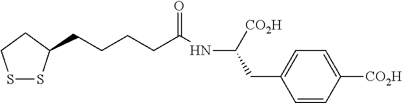 [SMILES: O=C(CCCC[C@@H]1CCSS1)N[C@@H](Cc1ccc(C(=O)O)cc1)C(=O)O] |
| AC | 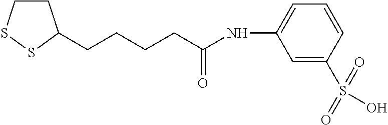 [SMILES: O=C(CCCCC1CCSS1)Nc1cccc(S(=O)(=O)O)c1] | AC′ | 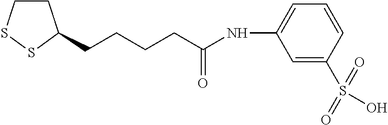 [SMILES: O=C(CCCC[C@@H]1CCSS1)Nc1cccc(S(=O)(=O)O)c1] |
| AD | 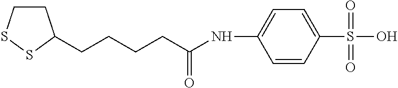 [SMILES: O=C(CCCCC1CCSS1)Nc1ccc(S(=O)(=O)O)cc1] | AD′ | 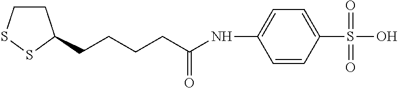 [SMILES: O=C(CCCC[C@@H]1CCSS1)Nc1ccc(S(=O)(=O)O)cc1] |
| AE | 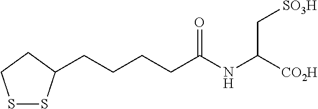 [SMILES: O=C(CCCCC1CCSS1)NC(CS(=O)(=O)O)C(=O)O] | AE′ | 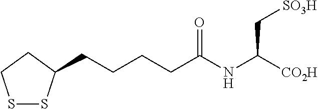 [SMILES: O=C(CCCC[C@@H]1CCSS1)N[C@@H](CS(=O)(=O)O)C(=O)O] |
| AF | 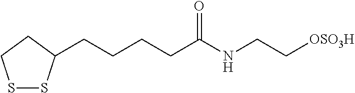 | AF′ | 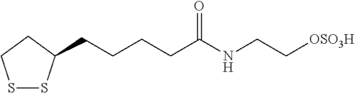 |
| AG | 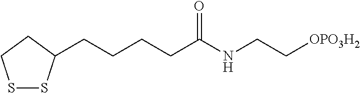 | AG′ | 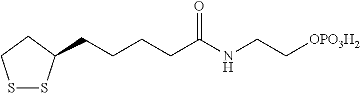 |
| AH | 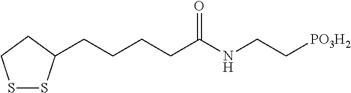 | AH′ | 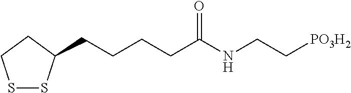 |
| AI | 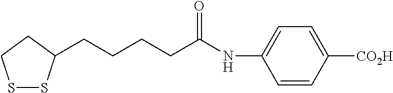 [SMILES: O=C(CCCCC1CCSS1)Nc1ccc(C(=O)O)cc1] | AI′ | 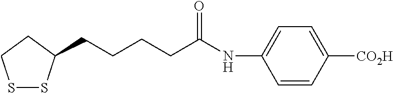 [SMILES: O=C(CCCC[C@@H]1CCSS1)Nc1ccc(C(=O)O)cc1] |

In a first aspect of the 2^{nd} specific embodiment of the present invention, the (R)-lipoyl stereoisomer of any of the compounds in Table A is substantially separated from the (S)-lipoyl stereoisomer(s).

In a 3^{rd} specific embodiment, the compound is represented by the following structural formula:

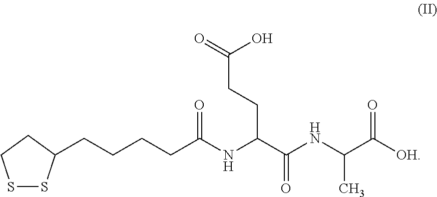 [SMILES: CC(NC(=O)C(CCC(=O)O)NC(=O)CCCCC1CCSS1)C(=O)O]

In a first aspect of the 3^{rd} embodiment, the compound is represented by the following structural formula:

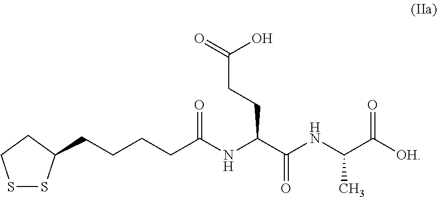 [SMILES: C[C@H](NC(=O)[C@H](CCC(=O)O)NC(=O)CCCC[C@@H]1CCSS1)C(=O)O]

In a second aspect of the 3^{rd} specific embodiment of the present invention, the (R)-lipoyl stereoisomer of the compound of Structural Formula II or IIa is substantially separated from the (S)-lipoyl stereoisomer(s).

Methods of making compounds of Structural Formula I, as well as details of their biological activities, are disclosed, for example, in International Publication No. WO2010/132657 and International Publication No. WO 2012/067947, the relevant teachings of which are incorporated by reference herein in their entirety.

### Formulations of the Invention

Provided herein are aqueous pharmaceutical formulations comprising a lipoyl compound that is substituted with at least one acidic substituent, such as a compound of Structural Formula I, Ia, II, IIa; and an inorganic base in an amount sufficient to deprotonate each acidic substituent in lipoyl compound, wherein the formulation has a pH of from about 6.5 to about 8.0 and a tonicity of from about 250 mOsm to about 350 mOsm.

“Aqueous pharmaceutical formulation” refers to a water-containing solution or suspension of sufficient purity and quality such that, when administered to a patient, such as a human or animal, the active ingredient(s) of the formulation typically exert a desired therapeutic effect (e.g., prevent the onset of; alleviate, partially or substantially or totally, the symptoms of; or delay, inhibit or stop the progression of a disorder or disease being treated). An aqueous pharmaceutical formulation should typically not produce an adverse reaction.

In some embodiments, a lipoyl compound is present in a formulation of the invention in a concentration of from about 5 mg/mL to about 50 mg/mL, from about 10 mg/mL to about 40 mg/mL, from about 9 mg/mL to about 30 mg/mL or of about 25 mg/mL.

In some embodiments, an aqueous pharmaceutical formulation comprising a lipoyl compound is substantially free of polymerized derivative(s) of the lipoyl compound. Lipoyl compounds have a propensity to form impurities, such as polymers, upon exposure to light to form polymerized derivatives of lipoyl compounds. Although not wishing to be bound by any particular theory, the formation of impurities, such as polymeric derivative(s) of lipoyl compounds, is proposed to proceed by photolytic opening of the dithiolane ring resulting in a diradical, followed by propagation through intermolecular disulfide bond formation.

“Polymerized derivative(s) of the lipoyl compound” refers to derivative(s) of a lipoyl compound that contain two or more lipoyl moieties. In some cases, the polymerized derivatives have a molecular weight of greater than about 3,500 Daltons.

“Substantially free” means that a formulation contains less than about 5% of an indicated impurity or indicated impurities in the formulation (e.g., polymerized derivative(s) of the lipoyl compound in the formulation, lipoic acid). In some embodiments, a formulation is substantially free of all impurities (e.g., polymerized derivative(s) of the lipoyl compound in the formulation and lipoic acid). Impurities can be represented by a single chemical species (e.g., (R)-lipoic acid) or several different chemical species (e.g., polymerized derivatives of the lipoyl compound in the formulation, (R)-lipoic acid, etc.). For example, a formulation can contain less than about 4%, less than about 3%, less than about 2%, less than about 1%, less than about 0.5%, less than about 0.25% or less than about 0.1% of an indicated impurity or indicated impurities (e.g., polymerized derivative(s) of the lipoyl compound in the formulation) or all impurities (e.g., polymerized derivative(s) of the lipoyl compound in the formulation and lipoic acid). Preferably, the formulation contains less than about 1%, preferably, less than about 0.5%, more preferably, less than about 0.25%, yet more preferably, less than about 0.1% of polymerized derivative(s) of the lipoyl compound in the formulation.

The purity of a formulation comprising a lipoyl compound can be assessed in terms of the amount (e.g., concentration) of desired lipoyl compound (including stereoisomers of the desired lipoyl compound) in the formulation compared to the amount(s) of impurities in the formulation. The measurement of the purity of a formulation comprising a lipoyl compound is a measurement distinct from the measurement of the stereopurity of the lipoyl compound in the formulation. Impurities include, but are not limited to, other lipoyl-containing compounds of different structural formulas (e.g., polymerized derivative(s) of the lipoyl compound, other lipoyl compounds). Typically, the purity of a formulation comprising a lipoyl compound is assessed in terms of the amount of the lipoyl compound compared to the amount of other lipoyl-containing compounds of different structural formulas. The purity of a formulation disclosed herein can be at least or about 95%, at least or about 98%, at least or about 99%, at least or about 99.5% or at least or about 99.9%. The purity of a formulation or the amount of impurities in a formulation can be measured, for example, using the assay of chemical purity described in the Exemplification.

“Inorganic base,” as used herein, includes both bases that contain no carbon atom and inorganic carbon bases that contain carbon-carbon or carbon-hydrogen bond(s), but not both. The choice of inorganic base is not particularly limited, except that the base should be able to deprotonate an acidic substituent in a lipoyl compound. Exemplary inorganic bases include sodium hydroxide, potassium hydroxide, calcium hydroxide, magnesium hydroxide, cesium hydroxide, lithium hydroxide, sodium carbonate, potassium carbonate, calcium carbonate, cesium carbonate and sodium bicarbonate. Sodium hydroxide is a particularly preferred inorganic base.

In some embodiments, the inorganic base is a sodium base. “Sodium base” refers to any inorganic sodium salt that dissociates in aqueous solution into a sodium cation and an anion capable of deprotonating an acidic substituent in a lipoyl compound. Exemplary sodium bases include sodium hydroxide, sodium carbonate and sodium bicarbonate.

In some embodiments, the inorganic base is a hydroxide base. “Hydroxide base” refers to any inorganic base, typically an ionic base (e.g., a salt), that dissociates in aqueous solution into a hydroxide anion and a cation. Exemplary hydroxide bases include sodium hydroxide, potassium hydroxide, calcium hydroxide, magnesium hydroxide, cesium hydroxide, lithium hydroxide, etc. Preferred hydroxide bases include sodium hydroxide, potassium hydroxide and calcium hydroxide.

It is understood that, upon formulation in an aqueous solution comprising a sufficient amount of inorganic base, a compound possessing an acidic substituent, for example, a compound of Structural Formula II, will become deprotonated. As such, the compound of Structural Formula II no longer exists, per se, but forms perhaps an ion pair with a cation formed upon dissolution of the inorganic base in the aqueous solution. The formulations described herein are meant to encompass this phenomenon and include the species formed as a result of formulating the listed elements into an aqueous pharmaceutical formulation.

As used herein, “acidic substituent” refers to any of the following functional groups in a compound described herein: —CO₂H, —SO₃H, —PO₃H₂, —OSO₃H, —OPO₃H₂, —B(OH)₂ and —NHOH. Thus, a compound of Structural Formula II contains two acidic substituents, one in the portion of the compound of Structural Formula I designated as variable X and one in the portion of a compound of Structural Formula I designated as R or W.

“An amount sufficient to deprotonate each acidic substituent” refers to an amount of an inorganic base (e.g., sodium base, hydroxide base, sodium hydroxide) approximately equal to or greater than the molar equivalents of the acidic substituents in a compound described herein. A compound of Structural Formula II contains two acidic substituents. Therefore, an amount sufficient to deprotonate each acidic substituent in a compound of Structural Formula II is an amount approximately equal to or greater than two molar equivalents of a compound of Structural Formula II. In other words, at least about two molar equivalents of an inorganic base should be present in an aqueous pharmaceutical formulation of the invention in order to deprotonate the acidic substituents present in a compound of Structural Formula II. Preferably, a formulation comprises an inorganic base in an amount that is greater than the molar equivalents of the acidic substituent(s) in a lipoyl compound.

In some embodiments, a formulation comprises inorganic base (e.g., sodium base, hydroxide base, sodium hydroxide) in a concentration of from about 25 mg/mL to about 200 mg/mL, from about 50 mg/mL to about 150 mg/mL or of about 125 mg/mL.

“Tonicity” is the effective osmolality of a solution and is equal to the sum of the concentrations of the solutes which have the capacity to exert an osmotic force across a membrane, such as a cell membrane. Osmolality is the measure of the number of osmoles of solute per kilogram of solvent in a solution. The pharmaceutical formulations described herein can be isotonic, hypotonic or hypertonic. Typically, the aqueous pharmaceutical formulations described herein are isotonic. Isotonic formulations are formulations that have essentially the same osmotic pressure as human blood, for example, osmotic pressure of from about 260 mOsm to about 320 mOsm. Slightly hypotonic formulations having a slightly lower osmotic pressure, for example, osmotic pressure of from about 250 mOsm to less than 260 mOsm. Slightly hypertonic formulations have a slightly higher osmotic pressure, for example, osmotic pressure of greater than 320 to about 350. Methods of measuring tonicity are well-known in the art and include melting point depression.

In some embodiments, an aqueous pharmaceutical formulation has a tonicity of from about 250 mOsm to about 350 mOsm. Preferably, an aqueous pharmaceutical formulation has a tonicity of from about 260 mOsm to about 320 mOsm.

A tonicity agent can be used to achieve and/or maintain the tonicity of an aqueous pharmaceutical formulation. Thus, in some embodiments, an aqueous pharmaceutical formulation further comprises a tonicity agent. When present, a tonicity agent should be present in a formulation in an amount sufficient to achieve and/or maintain a tonicity of from about 250 mOsm to about 350 mOsm or, preferably, from about 260 mOsm to about 320 mOsm. When present, a tonicity agent is preferably present at levels that are in accordance with the Food and Drug Administration's Inactive Ingredient Database for IV formulations. A tonicity agent can be non-ionic or ionic. Exemplary non-ionic tonicity agents include polyols, such as glycerin, glycerol, mannitol or erythritol; amino acids; and sugars, such as dextrose. Ionic tonicity agents include sodium chloride and potassium chloride.

In some embodiments, a formulation comprises a tonicity agent (e.g., an ionic tonicity agent such as sodium chloride) in a concentration of from about 1 mg/mL to about 10 mg/mL, from about 2.5 mg/mL to about 7.5 mg/mL, from about 3.5 mg/mL to about 6 mg/mL or of about 6 mg/mL.

In some embodiments, an aqueous pharmaceutical formulation further comprises a buffer. When present, a buffer should be present in a formulation in an amount sufficient to achieve and/or maintain a pH of from about 6.5 to about 8.0, preferably, from about 6.8 to about 7.6, more preferably, from about 7.0 to about 7.2. Exemplary buffers include phosphate, phosphate-buffered saline, succinate, gluconate, histidine, citrate, MES, ADA, PIPES, ACES, MOPSO, cholamine chloride, MOPS, BES, TES, HEPES, DIPSO, acetamidoglycine, TAPSO, POPSO, HEPPSO, HEPPS, tricine, glycinamide, bicine and TAPS. A particularly preferred buffer is phosphate buffer, for example, sodium phosphate buffer or sodium phosphate dibasic.

In some embodiments, a formulation comprises a buffer (e.g., phosphate buffer, such as sodium phosphate dibasic) in a concentration of from about 0.5 mg/mL to about 5 mg/mL, from about 1 mg/mL to about 3 mg/mL, from about 1.4 mg/mL to about 2.7 mg/mL or of about 1.4 mg/mL.

In some embodiments of a formulation of the invention, the formulation comprises from about 5 mg/mL to about 50 mg/mL of a lipoyl compound; and from about 25 mg/mL to about 200 mg/mL sodium hydroxide. The formulation has a pH of from about 7.0 to about 7.2 and a tonicity of from about 260 mOsm to about 320 mOsm. In a more specific embodiment, the formulation comprises from about 9 mg/mL to about 30 mg/mL of a compound for use in the formulations of the invention, and from about 50 mg/mL to about 150 mg/mL sodium hydroxide; and has a pH of from about 7.0 to about 7.2 and a tonicity of from about 260 mOsm to about 320 mOsm.

Also provided herein are aqueous pharmaceutical formulations comprising from about 5 mg/mL to about 30 mg/mL of a compound represented by Structural Formula IIa; from about 25 mg/mL to about 200 mg/mL sodium hydroxide; buffer; and a tonicity agent. The formulations have a pH from about 6.8 to about 7.6 and a tonicity of from about 260 mOsm to about 320 mOsm. Concentrations and alternative concentrations for the components of this formulation, as well as particular buffers and tonicity agents and alternative pH and tonicity ranges, are as described and defined hereinabove. For example, in one aspect of this embodiment, the formulation comprises about 25 mg/mL of the compound of Structural Formula IIa; about 125 mg/mL sodium hydroxide; sodium phosphate buffer; and sodium chloride, wherein the formulation has a pH of from about 7.0 to about 7.2 and a tonicity of from about 260 mOsm to about 320 mOsm.

The aqueous pharmaceutical formulations described herein are typically intended for parenteral (e.g., intraarticular, intramuscular, intravenous, intraventricular, intraarterial, intrathecal, subcutaneous, or intraperitoneal) and, in particular, intravenous administration. In some embodiments, the formulations described herein can be described as intravenous aqueous pharmaceutical formulations. The pharmaceutical formulations can be transferred, preferably aseptically, into an appropriate container, for example, an amber vial to provide a suitable dosage of the lipoyl compound. Suitable intravenous dosages of a lipoyl compound in a formulation of the invention can be from about 0.001 mg/kg to about 100 mg/kg, from about 0.01 mg/kg to about 100 mg/kg, from about 0.1 mg/kg to about 10 mg/kg, from about 1 mg/kg to about 5 mg/kg or from about 2 mg/kg to about 3 mg/kg body weight per treatment.

### Processes for Preparing Formulations of the Invention

Also provided herein is a process for preparing an aqueous pharmaceutical formulation (e.g., an aqueous pharmaceutical formulation having a pH of from about 6.5 to about 8.0 and a tonicity of from about 250 mOsm to about 350 mOsm) comprising a lipoyl compound (Structural Formula I, Ia, II, IIa, etc.). The process comprises providing a lipoyl compound (which is substituted with at least one acidic substituent); and providing an aqueous solution comprising an inorganic base in an amount sufficient to deprotonate each acidic substituent in the lipoyl compound. The volume of the aqueous solution is equal to or greater than about 75% of the volume of the formulation. The lipoyl compound is added to the aqueous solution, thereby forming a pharmaceutical solution, and the pharmaceutical solution is diluted to the volume of the formulation with a diluent to thereby prepare the aqueous pharmaceutical formulation. Concentrations and alternative concentrations for the components of this formulation, as well as particular lipoyl compounds and inorganic bases, alternative pH and tonicity ranges and additional components and concentrations thereof, are as described and defined hereinabove with respect to formulations.

A variety of diluents can be used to dilute the pharmaceutical solution, provided that the diluent forms, in combination with the other elements in the formulation, a pharmaceutically acceptable formulation. For example, if the formulation comprises buffer, an aliquot of the buffer can be employed for the dilution. Alternatively, an aliquot of the aqueous solution can be used as the diluent. Preferably, the diluent is water.

In some embodiments of the processes for preparing formulations of the invention, the formulation comprising a lipoyl compound is substantially free of polymerized derivatives of the lipoyl compound, wherein “substantially free” is as described above with respect to formulations of the invention.

In some embodiments of the processes for preparing formulations of the invention, the process further comprises adjusting the pH of the aqueous solution and/or pharmaceutical solution and/or pharmaceutical formulation. Often, it is convenient to adjust the pH after addition of the lipoyl compound to the aqueous solution, particularly when the lipoyl compound is substituted with one or more acidic substituents. For example, it is often necessary to basify a pharmaceutical solution in order to form a formulation having a pH in the recited ranges. pH adjustment, in particular, basification, can be done, for example, by adding a sufficient amount of sodium hydroxide to an aqueous solution and/or a pharmaceutical solution and/or a pharmaceutical formulation to form a formulation having the desired pH. Conversely, an acid, such as hydrochloric acid, can be added to an aqueous solution and/or pharmaceutical solution and/or pharmaceutical formulation to acidify the solution and thereby form a formulation having the desired pH.

In some embodiments of the processes for preparing formulations of the invention, the volume of the aqueous solution is equal to or greater than about 80%, equal to or greater than about 85%, equal to or greater than about 90% or equal to or greater than about 95% of the volume of the formulation.

In some embodiments, the aqueous solution further comprises a buffer, for example, phosphate buffer. When present, the amount of buffer in the aqueous solution is an amount sufficient (either alone or in combination with other components of the formulation, such as the diluent) to achieve a formulation having the desired concentration of buffer. Alternative buffers as well as exemplary concentrations of buffer are as described and defined hereinabove with respect to the formulations.

In some embodiments, the aqueous solution further comprises a tonicity agent, for example, an ionic tonicity agent such as sodium chloride. When present, the amount of tonicity agent in the aqueous solution is an amount sufficient (either alone or in combination with other components of the formulation) to form a formulation comprising the desired concentration of tonicity agent. Alternative tonicity agents as well as exemplary concentrations of tonicity agents are as described and defined hereinabove with respect to the formulations.

In some embodiments of the processes for preparing formulations of the invention, the process further comprises adjusting the tonicity of the aqueous solution and/or pharmaceutical solution and/or pharmaceutical formulation. Tonicity can be adjusted, for example, by adding a tonicity agent to the aqueous solution, for example, in an amount (either alone or in combination with other components of the formulation) sufficient to form a formulation comprising the desired concentration of tonicity agent.

In some embodiments, the aqueous pharmaceutical formulation has a pH of from about 6.5 to about 8.0 and a tonicity of from about 250 mOsm to about 350 mOsm.

It is understood that because the formulations are made by adding an appropriate amount of a lipoyl compound to an aqueous solution having a volume equal to or greater than 75% of the volume of the formulation, the concentration of each component of the aqueous or pharmaceutical solution or, indeed, the pH or tonicity of the aqueous or pharmaceutical solution, is likely not the concentration (or pH or tonicity) of the pharmaceutical formulation. One of skill in the art will understand that the factor of dilution, as well as the features of the diluent, should be taken into account when formulating the aqueous and pharmaceutical solutions. Thus, in order to formulate a formulation comprising from about 5 mg/mL to about 50 mg/mL lipoyl compound, the lipoyl compound is added to the aqueous solution in an amount sufficient to form a formulation comprising from about 5 mg/mL to about 50 mg/mL. For example, if the volume of the aqueous solution is 90% of the volume of the formulation and a concentration of lipoyl compound of 10 mg/mL is desired, 10 g of lipoyl compound should be added to, for example, 900 mL of aqueous solution, such that dilution with 100 mL of diluent will result in a formulation having 10 mg/mL lipoyl compound.

In some embodiments, the lipoyl compound is added to the aqueous solution in an amount sufficient to form a formulation comprising from about 5 mg/mL to about 50 mg/mL, from about 10 mg/mL to about 40 mg/mL, from about 9 mg/mL to about 30 mg/mL or about 25 mg/mL of the lipoyl compound.

In some embodiments, the amount of inorganic base in the aqueous solution is an amount sufficient (either alone or in combination with other components of the formulation, such as the diluent) to form a formulation comprising from about 25 mg/mL to about 200 mg/mL or from about 50 mg/mL to about 150 mg/mL inorganic base. Preferably, the amount of inorganic base in the aqueous solution is the amount sufficient to form a formulation having the desired concentration of inorganic base. For example, the aqueous solution comprises a sufficient amount of inorganic base such that additional inorganic base need not be included in the diluent to achieve a formulation comprising the desired concentration of inorganic base.

In some embodiments, the lipoyl compound is added to the aqueous solution in an amount sufficient to form a formulation comprising from about 5 mg/mL to about 50 mg/mL of the lipoyl compound; the inorganic base is sodium hydroxide and the amount of sodium hydroxide in the aqueous solution is an amount sufficient to form a formulation comprising from about 25 mg/mL to about 200 mg/mL sodium hydroxide; and the formulation has a pH of from about 7.0 to about 7.2 and a tonicity of from about 260 mOsm to about 320 mOsm. In a specific aspect of this embodiment, the lipoyl compound is added to the aqueous solution in an amount sufficient to form a formulation comprising from about 9 mg/mL to about 30 mg/mL of the lipoyl compound; and the amount of sodium hydroxide in the aqueous solution is an amount sufficient to form a formulation comprising from about 25 mg/mL to about 200 mg/mL sodium hydroxide.

Also provided herein is a process for preparing an aqueous pharmaceutical formulation comprising a compound of Structural Formula IIa. The process comprises providing an aqueous solution comprising a buffer, a tonicity agent and sodium hydroxide; adding a compound represented by Structural Formula IIa to the aqueous solution in an amount sufficient to form a formulation comprising from about 5 mg/mL to about 50 mg/mL of the compound of Structural Formula IIa, thereby forming a pharmaceutical solution; and diluting the pharmaceutical solution to the volume of the formulation with a diluent, thereby preparing the aqueous pharmaceutical formulation having a pH from about 6.8 to about 7.6 and a tonicity of from about 260 mOsm to about 320 mOsm. The amount of sodium hydroxide in the aqueous solution is an amount sufficient to form a formulation comprising from about 25 mg/mL to about 200 mg/mL sodium hydroxide and the volume of the aqueous solution is equal to or greater than about 75% of the volume of the formulation. Concentrations and alternative concentrations for the components of the formulation in this process, as well as particular buffers and tonicity agents and alternative pH and tonicity ranges, are as described and defined hereinabove. For example, in an aspect of this embodiment, the aqueous solution comprises sodium phosphate buffer, sodium chloride and sodium hydroxide. Compound of Structural Formula IIa is added to the aqueous solution in an amount sufficient to form a formulation comprising about 25 mg/mL compound of Structural Formula IIa and the amount of sodium hydroxide in the aqueous solution is an amount sufficient to form a formulation comprising about 125 mg/mL sodium hydroxide.

Also provided herein is an aqueous pharmaceutical formulation made according to any of the processes described herein.

### EXEMPLIFICATION

### Assay for Chemical Purity

The purity of a lipoyl compound was analyzed by reversed-phase high performance liquid chromatography (HPLC). Mobile phase A was 0.1% triflouroacetic acid (TFA) in H₂O and mobile phase B was 0.1% TFA in acetontrile (ACN). A linear gradient of 85:15 to 26:74 over 22 minutes at a flow rate of 1.5 mL/min at 30° C. was used. A lipoyl compound was diluted in ethanol:water (1:1) at a concentration of 1 mg/mL and a 25 μL aliquot was injected onto the HPLC. The sample was detected at 220 nm.

### Example 1

### Synthesis of (R)Lip-EA-OH

RLipoic acid (RLip-OH—50.0 g-0.242 mol) was added to a 2 L round-bottomed flask containing 500 mL acetone and mixed with magnetic stirring until dissolved. The solution was protected from direct light by covering the flask with foil. N,N-Diisopropylethylamine (DIEA—42.2 mL-0.242 mol) and N,N′-disuccinimidylcarbonate (DSC—77.6 g-0.303 mol) were added sequentially and the reaction was stirred vented for 3 hours at room temperature.

Glutamyl-alanine (H-EA-OH—66.1 g-0.303 mol) followed by DIEA (52.8 mL-0.303 mol) were added to a separate 1 L Schott bottle containing 330 mL water and mixed with a magnetic stirrer until dissolved. The H-EA-OH solution was rapidly added to the activated lipoic acid solution in the 2 L round-bottomed flask. The pH of this coupling reaction was initially 7.0. The pH was monitored and maintained between 6.9 and 7.0 by the addition of DIEA. The amount of DIEA added was 34 mL (0.354 mol) over 40 minutes, at which point the pH of the coupling reaction stabilized. The reaction was stirred overnight at room temperature while vented and protected from direct light.

After stirring overnight, water (250 mL) was added to the reaction mixture and the solution was transferred to a reparatory funnel. Isopropyl acetate (IpOAc—500 mL) was added and the solution mixed, and then allowed to separate. The organic layer was removed and the product-containing aqueous layer was washed with an additional 500 mL IpOAc. The aqueous product-containing solution was transferred to a 4 L Erlenmeyer flask. Isopropyl alcohol (IPA—190 mL) and IpOAc (1060 mL) were added to the flask. The solution was rapidly stirred and acidified by the slow addition of 0.25 N sulfuric acid, then 0.5 N sulfuric acid, until the pH was measured at 2.0. The combined solution was transferred to a reparatory funnel and allowed to settle. The aqueous solution was removed and the product-containing organic solution was washed one time with water (250 mL).

The product-containing organic solution was passed through a medium porosity fritted glass filter and transferred to a round-bottomed flask. The volume of the solution was reduced on a rotary evaporator with a bath temperature of 43° C. After 600 mL of the organic solution had been removed, an additional 600 mL of IpOAc was charged to the 2 L flask and the solution again reduced in volume on the rotary evaporator. Product began to crystallize from solution after the removal of approximately 100 mL. The evaporation was halted and the product crystallized overnight from the spinning flask as it cooled to room temperature. Solid RLip-EA-OH was collected by filtration and washed two times with 100 mL IpOAc.

The product was immediately recrystallized by dissolving the isolated RLip-EA-OH wet cake in a prepared mixed solution of IPA (190 mL), IpOAc (1000 mL), and water (60 mL). This solution was passed through a medium porosity fitted glass filter and transferred to a round-bottomed flask. The volume of the solution was reduced on a rotary evaporator with a bath temperature of 42° C. After 650 mL of the organic solution had been removed, an additional 650 mL of IpOAc was charged to the 2 L flask and the solution again reduced in volume on the rotary evaporator. Product began to crystallize from solution after the removal of approximately 600 mL. The evaporation was halted and the product crystallized overnight from the spinning flask as it cooled to room temperature. Solid RLip-EA-OH was collected by filtration and washed two times with 100 mL IpOAc and dried for 2 days at 40° C. under vacuum.

(R)Lip-EA-OH was analyzed by HPLC using the assay for purity described above. RLip-EA-OH was isolated in a 38% overall yield (47.2 g-0.116 mol) at >98% purity (HPLC area percent).

### Example 2

### Process for Making a 30 mg/mL (R)Lip-EA-OH Formulation

The initial procedure used to formulate (R)Lip-EA-OH consisted of the following sequence:
1. Neutralize solid (R)Lip-EA-OH by treatment with aqueous sodium bicarbonate or sodium hydroxide;
2. Dilute with water and saline to near the final volume and concentration; and
3. Adjust pH and add water to final volume and concentration.

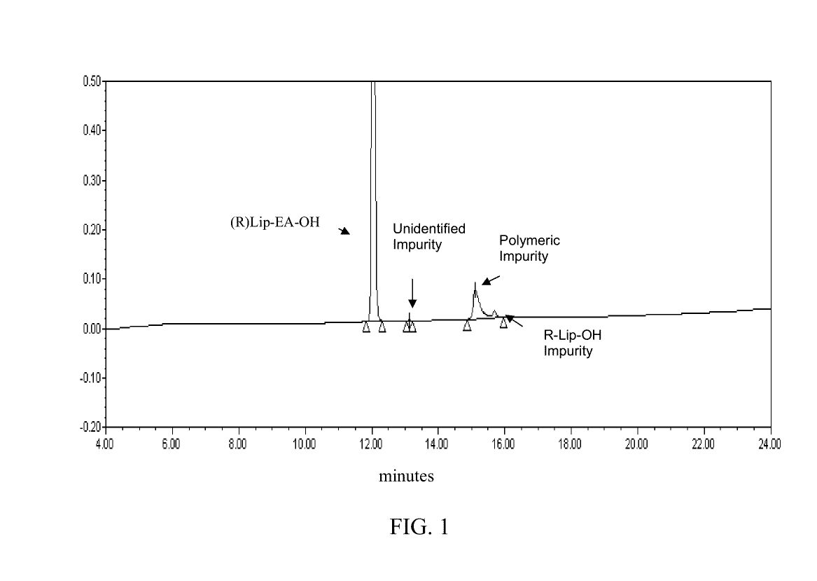

This formulation procedure was used effectively to prepare formulation from multiple lots of (R)Lip-EA-OH that were synthesized at scales below 50 g. As the synthetic process was scaled-up, however, partial polymerization of (R)Lip-EA-OH was observed during formulation. For example, (R)Lip-EA-OH, which initially contained <0.1% polymer by HPLC area count analysis, partially polymerized when formulated using the above procedure. Analysis of the HPLC chromatogram of the partially polymerized formulation indicated the formation of polymer at levels >20%, based upon HPLC area counts (see FIG. 1).

Since (R)Lip-EA-OH was converted to the corresponding bis-sodium salt in situ during formulation, a procedure for synthesizing the bis-sodium salt of (R)Lip-EA-OH was examined. (R)Lip-EA-OH treated with 2 equivalents of NaOH and refluxed in ethanol-water (19:1) formed a gummy solid. Analysis of the HPLC chromatogram indicated that the product contained a high level of polymer content. A pH neutral solution of the polymeric material was isolated by dialysis using a 3500 Dalton molecular weight selective membrane. The isolated polymer was treated with the reducing agent dithiothreitol (DTT), and the resulting sample was analyzed by HPLC. Analysis of the HPLC chromatograms indicated that DTT reduced the polymer to the corresponding disulfhydryl analog of (R)Lip-EA-OH containing the dihydrolipoyl moiety. These experiments suggested that preparation of the solid disodium salt of (R)Lip-EA-OH was not an optimal synthetic pathway.

As a result of the observed polymerization using the initial formulation protocol described above, an alternative formulation protocol was developed. In the revised protocol, (R)Lip-EA-OH was added to a previously compounded aqueous solution. The revised procedure avoided (R)Lip-EA-OH solutions of high concentration. The general revised formulation protocol consisted of the following sequence:
1. Prepare an aqueous solution that combines the required amounts of sodium bicarbonate or sodium hydroxide, water, and saline at near the total volume (>90%);
2. Add (R)Lip-EA-OH to the compounding vessel; and
3. Adjust the pH and dilute with water to final volume and concentration of formulation.

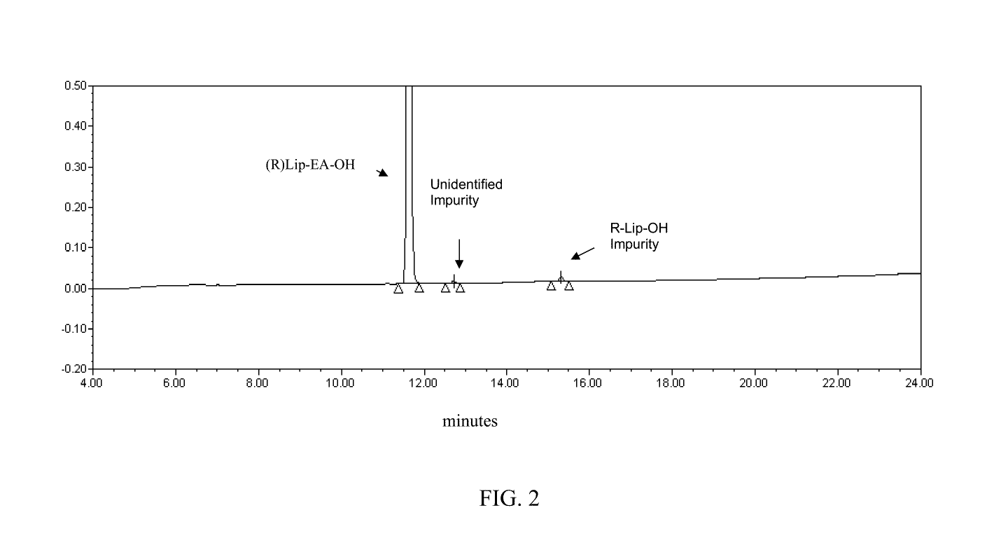

A 30 mg/mL (R)Lip-EA-OH formulation formulated according to the revised procedure contained <0.1% polymer by analysis of the HPLC area counts. Analysis of the HPLC chromatogram indicated that polymerization of (R)Lip-EA-OH did not occur to any significant extent during formulation using the revised protocol (see FIG. 2).

Specifically, sodium chloride (45 g), sodium phosphate dibasic (20.1 g), and 1 N NaOH (aq) (375 g) were added to an open stirred flask containing 6 L water. The solution was stirred until all components were dissolved. Powdered Lip-EA-OH (75 g) was slowly added to the flask and the solution mixed until complete dissolution. The solution pH was measured and adjusted to pH 7.0-7.2 with the addition of either 1 N NaOH (aq) or 1 N HCl (aq). The solution was diluted with the addition of water to a total volume of 7.5 L. The pH was again measured and adjusted to pH 7.0-7.2 with the addition of either 1 N NaOH (aq) or 1 N HCl (aq). Solution osmolality was measured, and the concentration of RLip-EA-OH in the formulation was confirmed by HPLC using the assay for chemical purity described above.

The resulting drug formulation was a pH neutral isotonic saline solution of (R)Lip-EA-OH. Since (R)Lip-EA-OH contains two carboxylic acid functions, treatment with an aqueous sodium bicarbonate or sodium hydroxide solution provides a highly water soluble bis-sodium salt of (R)Lip-EA-OH. Typically, formulations comprising up to about 30 mg/mL (R)Lip-EA-OH are isotonic, whereas formulation comprising greater than about 30 mg/mL (R)Lip-EA-OH are hypertonic.

### Example 3

### A 10 mg/ml (R)Lip-EA-OH Formulation

A 10 mg/mL (R)Lip-EA-OH formulation was prepared using the general revised formulation protocol described in Example 2. Details of the formulation are given in Table 1 and Table 2.

TABLE 1
10 mg/mL (R)Lip-EA-OH Formulation
Solution Properties.

| Properties | Value | |
|---|---|---|
| (R)Lip-EA-OH | 9.0-11.0 | mg/mL |
| Osmolality | 260-320 | mOsm |
| Phosphate | 10 | mmol |
| pH | 6.8-7.6 | |

TABLE 2
Composition of 10 mg/mL (R)Lip-EA-OH Formulation.

| Component | mg/mL | Concentration (%) |
|---|---|---|
| Sodium phosphate dibasic | 2.68 | 0.268 |
| heptahydrate, USP | | |
| Sodium chloride, USP | 6.0 | 0.6 |
| (R)Lip-EA-OH | 10 | 1 |
| 1N Sodium hydroxide solution | 50 | 5 |
| Sterile Water for Injection, USP | QS | >93 |

Stability data for a representative 7,500-mL batch of the 10 mg/mL (R)Lip-EA-OH formulation is shown in Table 2A and Table 2B.

TABLE 2A
10 mg/mL Stability Data for a 10 mg/mL (R)Lip-EA-OH Batch at 5 ± 3° C.

| | | Results | | | | | | | |
|---|---|---|---|---|---|---|---|---|---|
| | | Time Interval (Months) | | | | | | | |
| Test | Speci- | | | | | | 3 | | 4 |
| Description | fications | 0 | 0.5 | 1 | 2 | 3 | Upright † | 4 | Upright † |
| Appearance | Clear, no | Clear no | Clear no | Clear no | Clear no | Clear no | Clear no | Clear no | Clear no |
| (Product) | precipitate, | precipitate, | precipitate, | precipitate, | precipitate, | precipitate, | precipitate, | precipitate, | precipitate, |
| | note color | Light | Light | Light | Light | Light | Light | Light | Light |
| | | yellow | yellow | yellow | yellow | yellow | yellow | yellow | yellow |
| Appearance | No evidence | No | No | No | No | No | No | No | No |
| (Closure) | of leaking | evidence * | evidence * | evidence * | evidence * | evidence * | evidence * | evidence * | evidence * |
| | or crusting | | | | | | | | |
| Assay - | 9.0-11.0 | 9.9 | 9.7 | 9.4 | 9.6 | 10.0 | 9.9 | 9.6 | 9.6 |
| HPLC UV | mg/mL | | | | | | | | |
| detection | | | | | | | | | |
| (mg/mL) | | | | | | | | | |
| Osmolality | 260-320 | 284 | 284 | 289 | 286 | 286 | 287 | 284 | 284 |
| (mOsm/kg) | | | | | | | | | |
| pH | 6.8-7.6 | 7.1 | 7.2 | 7.2 | 7.2 | 7.6 | 7.2 | 7.2 | 7.1 |
| Sterility | No growth | No growth | NS | NS | NS | NS | NS | NS | NS |
| | within 14 | within 14 | | | | | | | |
| | days | days | | | | | | | |
| Endotoxins | NMT 5 | <0.06 | NS | NS | NS | NS | NS | NS | NS |
| | EU/mL | | | | | | | | |

| | | | | | | | | |
|---|---|---|---|---|---|---|---|---|
| Particulates (obscuration | | | | | | | | |
| USP Particulates Light | | | | | | | | |
| Obscuration | | | | | | | | |
| ≧10 μm NMT 6000 | 2688 | NS | NS | NS | NS | NS | NS | NS |
| per container | | | | | | | | |
| ≧25 μm NMT 600 | 11 | | | | | | | |
| per container | | | | | | | | |

† Vials were stored inverted. A set of vials were turned upright and tested at the 3 and 4 month time points. The limitation on the number of bottles placed under stability conditions did not permit for turning more bottles upright for testing at additional time periods.
^{#} Out of Specification
No evidence* = No evidence of leaking or crusting
NS = Not Scheduled

TABLE 2B
10 mg/mL Stability Data for a 10 mg/mL (R)Lip-EA-OH Batch at 5 ± 3° C. (continued).

| | | Results | | | | | |
|---|---|---|---|---|---|---|---|
| Test | Speci- | Time Interval (Months) | | | | | |
| Description | fications | 0 | 6 | 9 | 12 | 18 | 24 |
| Appearance | Clear, no | Clear no | Clear no | Clear no | Clear no | Clear no | Clear no |
| (Product) | precipitate, | precipitate, | precipitate, | precipitate, | precipitate, | precipitate, | precipitate, |
| | note color | Light | Light | Light | Light | Light | Light |
| | | yellow | yellow | yellow | yellow | yellow | yellow |
| Appearance | No evidence | No | No | No | No | No | No |
| (Product) | of leaking | evidence* | evidence* | evidence* | evidence* | evidence* | evidence* |
| | or crusting | | | | | | |
| Assay - | 9.0-11.0 | 9.9 | 9.7 | 9.7 | 9.6 | 9.5 | 9.3 |
| HPLC UV | mg/mL | | | | | | |
| detection | | | | | | | |
| (mg/mL) | | | | | | | |
| Osmolality | 260-320 | 284 | 285 | 286 | 286 | 280 | 284 |
| (mOsm/kg) | | | | | | | |
| pH | 6.8-7.6 | 7.1 | 7.2 | 7.0 | 7.1 | 7.2 | 7.2 |
| Sterility | No growth | No growth | No growth | NS | No growth | | No growth |
| | within 14 | within 14 | within 14 | | within 14 | | within 14 |
| | days | days | days | | days | | days |
| Endotoxins | NMT 5 | <0.06 | <0.06 | NS | <0.06 | | <0.06 |
| | EU/mL | | | | | | |

| | | | | | |
|---|---|---|---|---|---|
| Particulates (obscuration | | | | | |
| USP Particulates Light | | | | | |
| Obscuration | | | | | |
| ≧10 μm NMT 6000 | 2688 | 674 | NS | 575 | 2354 |
| per container | | | | | |
| ≧25 μm NMT 600 | 11 | 5 | | 1 | 22.2 |
| per container | | | | | |

^{#} Out of Specification
No evidence* = No evidence of leaking or crusting
NS = Not Scheduled

### Example 4

### A 25 mg/ml (R)Lip-EA-OH Formulation

A 25 mg/mL (R)Lip-EA-OH formulation was prepared using the general revised formulation protocol described in Example 2. Details of the formulation are given in Table 3.

TABLE 3
Composition of a 25 mg/mL (R)Lip-EA-OH Formulation

| | | Strength (label claim) | |
|---|---|---|---|
| Component and Quality | | Quantity | |
| Standard (and Grade, if | | per unit | Concentration |
| applicable) | Function | mg/mL | % |
| Dibasic Sodium phosphate, | Buffer | 1.42 | 0.142% |
| anhydrous, USP | | | |
| Sodium chloride, USP | Isotonicity | 3.5 | 0.35% |
| (R)Lip-EA-OH | API | 25 | 2.5% |
| 1N Sodium hydroxide solution | pH adjustment | 125.0 | 12.5% |
| Sterile Water for Injection, | | QS | >93 |
| USP | | | |
| Total | | | 100% |

A compatibility study was conducted to evaluate the effects of saline dilution and ambient light exposure using the 25 mg/mL (R)Lip-EA-OH dose formulation described in this example. The purpose of the study was to provide evidence of stability of the non-diluted formulation and a saline-diluted formulation for three hours in syringes under normal and ultraviolet light. These conditions follow conditions that were performed in the Phase 2 CARIN clinical trial. Syringes were filled with either 15 mL of 25 mg/mL (R)Lip-EA-OH dose formulation (undiluted DP) or 5 mL of 25 mg/mL (R)Lip-EA-OH dose formulation and 15 mL saline (diluted DP). Samples were analyzed for (R)Lip-EA-OH assay, related substances, and pH.

Results for (R)Lip-EA-OH assay, related substances and pH indicated that there was no loss of product quality. (R)Lip-EA-OH assay and pH for diluted and undiluted samples were within specification and no new related substances were detected. These results indicate that a syringe containing 0.9% saline diluent and 25 mg/mL (R)Lip-EA-OH dose formulation are compatible.

### Example 5

### A 25 mg/mL (R)Lip-EA-OH Batch Formulation

The components of the dosage form to be used in a manufacturing process, and their amounts on a per batch basis are provided in Table 4.

TABLE 4
Components and Amounts of the 25 mg/mL (R)Lip-EA-OH
Dosage Form Used in a Manufacturing Process.

| Strength (label claim) | 25 | mg/mL |
|---|---|---|
| Batch Size(s) (number of dosage units) | 14,000 | mL |
| Component and Quality Standard | | |
| (and Grade, if applicable) | Quantity per batch | |
| Dibasic Sodium phosphate, anhydrous, USP | 19.88 | g |
| Sodium chloride, USP | 49.00 | g |
| (R)Lip-EA-OH | 350.0 | g |
| 1N Sodium hydroxide solution | 1750 | g |
| Sterile Water for Injection, USP | 11200 | g initial, |
| | Sufficient quantity to bring | |
| | total mass to 14000 g. | |
| Total | 14000 | g |

The following manufacturing and packaging description was used to prepare an (R)Lip-EA-OH dose formulation. The formulation was prepared under cGMP (current good manufacturing practices) conditions.

The manufacturing of 25 mg/mL (R)Lip-EA-OH dose formulation follows the same process as 10 mg/mL (R)Lip-EA-OH dose formulation. The following components were added to an empty vessel with thorough mixing until dissolution in the following order:
1. Sterile water for injection (argon sparge while compounding)
2. Sodium chloride
3. Sodium phosphate dibasic
4. 1 N sodium hydroxide.

(R)Lip-EA-OH was then added to the vessel. The solution was stirred for 1 hour or until the solid dissolved. If required, the pH of the solution was adjusted using 1 N sodium hydroxide or 1 N hydrochloric acid to 7.1±0.1. Sterile water for injection (WFI) was added to final weight. Osmolality and pH were evaluated and, if required, the pH of the solution was adjusted using 1 N NaOH (aq) or 1 N HCl (aq) to 7.1±0.1. An aseptic transfer was performed followed by an aseptic fill consisting of 18 mL of (R)Lip-EA-OH dose formulation into individual 20 mL amber vials. The vials were purged with argon, sealed, inspected and then labeled.

### Example 6

### Efficacy of a 10 mg/mL (R)Lip-EA-OH Formulation

Acute myocardial infarction and acute stroke are manifestations of sudden occlusion of vessels of the coronary and cerebral circulations, respectively; the morbidity and mortality of these conditions are a direct manifestation of cell injury and death. Loss of cellular perfusion leading to cell injury and death is a common pathophysiologic mechanism for both natural disease states and operative procedures. Given the necessary interruption of blood flow to the heart during (on-pump) coronary bypass surgery and with major organ transplantation, ischemia-mediated cellular damage also frequently complicates these procedures. During percutaneous coronary intervention (PCI) and stent placement, both balloon inflation and downstream embolization due to dislodged plaque can lead to ischemic injury. Although the benefit of reperfusion therapy such as coronary artery bypass grafting (CABG) or PCI for ischemic heart disease is clear, reperfusion itself may result in deleterious effects, including cardiomyocyte death, microvascular injury, myocardial stunning, and arrhythmias. Emerging data suggest that distal embolization of atherothrombotic material accompanying balloon-induced plaque disruption results in impaired microcirculatory flow and ventricular dysfunction. When cardiac enzymes are measured after PCI, up to 30% of patients have elevated levels of CK-MB or other evidence of periprocedural myocardial injury, and similar proportions of patients develop electrocardiographic changes. Although the contribution of inflammation and endothelial injury is less clear, the final common pathway is potentially irreversible cardiomyocyte injury that manifests clinically as adverse events, including increased mortality.

A prospective, multi-center, blinded, randomized, placebo-controlled study to evaluate single dose regimens of a 10 mg/mL (R)Lip-EA-OH formulation for IV administration or single dose placebo in patients with stable coronary artery disease undergoing elective stent placement by PCI meeting all the eligibility criteria was conducted. The (R)Lip-EA-OH formulation was made in accordance with Example 3 and was administered at one of three doses: 0.8 mg/kg, 1.6 mg/kg and 2.4 mg/kg. The primary objective was to assess the safety of the (R)Lip-EA-OH formulation, ascertained by measuring the changes in CK-MB values up to 24 hours after the last balloon inflation. The secondary objective was to evaluate reduction of myocardial injury associated with stent PCI, as determined by serial measurements of cardiac biomarkers and as determined by continuous and serial ECG readings.

(R)Lip-EA-OH treatment demonstrated efficacy in reducing the myocardial injury and has shown cardioprotective action. The efficacy markers of periprocedural injury such as changes in levels of Troponin-T, CK-MB AUC₀₋₂₄ and Cₘₐₓ of CK-MB were lowest in the 2.4 mg/kg dose group.

Efficacy.

The primary outcome measure was the change in CK-MB at 24 hours after the last balloon inflation and it served as a surrogate marker for myocardial injury considered for safety and efficacy. CK-MB levels were measured on Day 1 at 0, 6, 12, 18, and 24 hours after the last balloon inflation.

The mean change from baseline of CK-MB values at 24 hours was highest in the placebo group (3.14 ng/mL) as compared to all the test groups. The mean change from baseline of CK-MB values at 24 hours for 0.8, 1.6 and 2.4 mg/kg dose groups was 1.80, 2.54, and 0.44 ng/mL, respectively. Since the positive mean change in CK-MB value from baseline is indicative of myocardial injury, the 2.4 mg/kg dose group in the test arm showed least myocardial injury following PCI, followed by the 0.8 mg/kg dose, 1.6 mg/kg dose and placebo arm, respectively. The changes from baseline for all four groups at all post-procedure time points are shown in Table 5. A comparison of the difference in the change from baseline at 24 hours between the placebo and 2.4 mg/kg dose groups showed a strong statistical trend (p=0.0505).

TABLE 5
Summary of CK-MB (ng/mL) - Change from Baseline.

| | | Treatment | | | | |
|---|---|---|---|---|---|---|
| Time | | | (R)Lip-EA-OH | | | Between |
| Point | | Placebo | 0.8 mg/kg | 1.6 mg/kg | 2.4 mg/kg | Groups |
| (hr) | Statistic | (N = 35) | (N = 36) | (N = 35) | (N = 36) | p-value (2] |
| 6 | N | 30 | 31 | 28 | 31 | |
| | Mean | 0.55 | 0.12 | 0.76 | −0.10 | 0.1396 |
| | Std Dev | 1.792 | 0.737 | 2.407 | 0.759 | |
| | Within Group | 0.7170 | 0.9239 | 0.4324 | 0.5286 | |
| | p-Value (1] | | | | | |
| | Between Group | | | | 0.0684 | |
| | p-Value (3] | | | | | |
| 12 | N | 32 | 30 | 29 | 31 | |
| | Mean | 2.16 | 1.63 | 1.95 | 0.10 | 0.3449 |
| | Std Dev | 6.980 | 5.074 | 4.407 | 1.178 | |
| | Within Group | 0.0787 | 0.0418 | 0.0227 | 0.6567 | |
| | P-Value (1] | | | | | |
| | Between Group | | | | 0.1108 | |
| | p-Value (3] | | | | | |
| 18 | N | 31 | 31 | 29 | 32 | |
| | Mean | 2.73 | 1.87 | 2.55 | 0.30 | 0.3508 |
| | Std Dev | 9.008 | 5.041 | 5.721 | 1.156 | |
| | Within Group | 0.0281 | 0.0171 | 0.0032 | 0.1142 | |
| | p-Value (1] | | | | | |
| | Between Group | | | | 0.1343 | |
| | p-Value (3] | | | | | |
| 24 | N | 31 | 33 | 29 | 32 | |
| | Mean | 3.14 | 1.80 | 2.54 | 0.44 | 0.1788 |
| | Std Dev | 7.565 | 4.634 | 4.853 | 1.250 | |
| | Within Group | 0.0123 | 0.0162 | 0.0033 | 0.0271 | |
| | p-Value (1] | | | | | |
| | Between Group | | | | 0.0505 | |
| | p-Value (3] | | | | | |

N = Number of subjects with CKMB at the given time point. Baseline is the value from the Screening visit.
(1] p-values from a repeated measures ANOVA model on the with-in group change from baseline values with a term for time point. In essence this is a paired t-test that takes into account the repeated measurements within a subject.
(2] p-value from an ANOVA model, Dunnett's test comparing the active treatment groups with placebo as control.
(3] Between groups p-value for placebo and (R)Lip-EA-OH 2.4 mg/kg groups based on t-test for means.

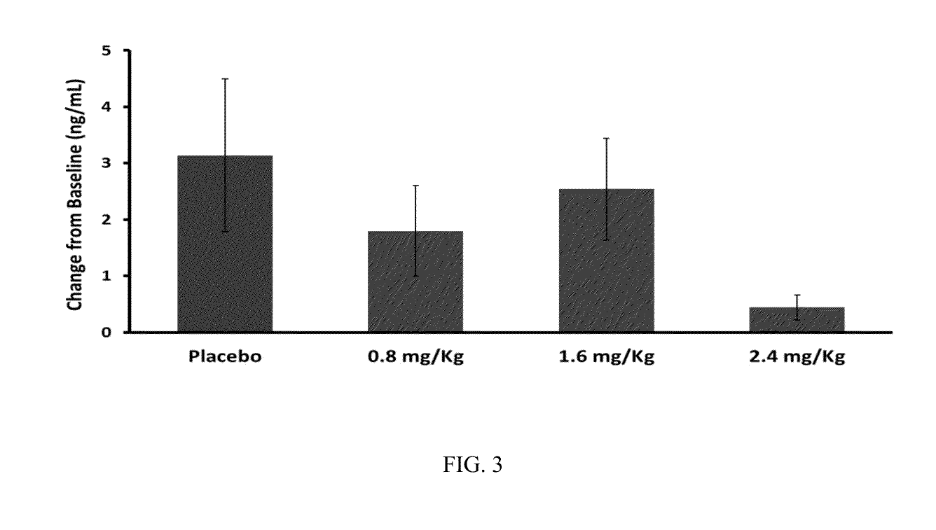

The maximum serum concentration of CK-MB (Cₘₐₓ) is calculated by subtracting the baseline value from the maximum concentration of CK-MB measured at any time point. CK-MB Cₘₐₓ is correlated to the extent of myocardial injury associated with the PCI. The greatest Cₘₐₓ value observed was in the placebo group. Using Cₘₐₓ as an indicator of injury, the greatest injury was observed in the placebo group while the least amount of injury was observed in the 2.4 mg/kg dose group, followed by the 0.8 mg/kg dose and 1.6 mg/kg dose groups, respectively. A comparison of the Cₘₐₓ difference between the placebo and 2.4 mg/kg dose groups showed a strong statistical trend (p=0.0616) (FIG. 3).

The mean baseline values of Troponin-T (TnT) were 0.001, 0.001, 0.001, and 0.003 ng/mL in the 0.8, 1.6, and 2.4 mg/kg and placebo groups, respectively. At 24 hours, these values changed to 0.042, 0.066, 0.019, and 0.135 ng/mL in the 0.8, 1.6, and 2.4 mg/kg and placebo groups, respectively (Table 6).

TABLE 6
Summary of Evaluation Parameters-Troponin-T (ng/mL).

| | | Treatment | | | |
|---|---|---|---|---|---|
| | | | (R)Lip-EA-OH | | |
| | | Placebo | 0.8 mg/kg | 1.6 mg/kg | 2.4 mg/kg |
| Day | Statistic | (N = 35) | (N = 36) | (N = 35) | (N = 36) |
| Baseline (Screening) | n | 32 | 33 | 29 | 33 |
| | Mean | 0.003 | 0.001 | 0.001 | 0.001 |
| | Std Dev | 0.009 | 0.007 | 0.004 | 0.005 |
| | Median | 0.000 | 0.000 | 0.000 | 0.000 |
| | Range | 0.000-0.040 | 0.000-0.040 | 0.000-0.020 | 0.000-0.020 |
| Day 1 (24 hours) | n | 31 | 33 | 30 | 32 |
| | Mean | 0.135 | 0.042 | 0.066 | 0.019 |
| | Std Dev | 0.286 | 0.122 | 0.183 | 0.039 |
| | Median | 0.020 | 0.000 | 0.000 | 0.000 |
| | Range | 0.000-1.160 | 0.000-0.670 | 0.000-0.940 | 0.000-0.150 |

N = Number of subjects with Troponin-T at the given time point. At Baseline, most of the measurements were below the level of detection (which is 0.001). Hence, statistics are shown with three digits.

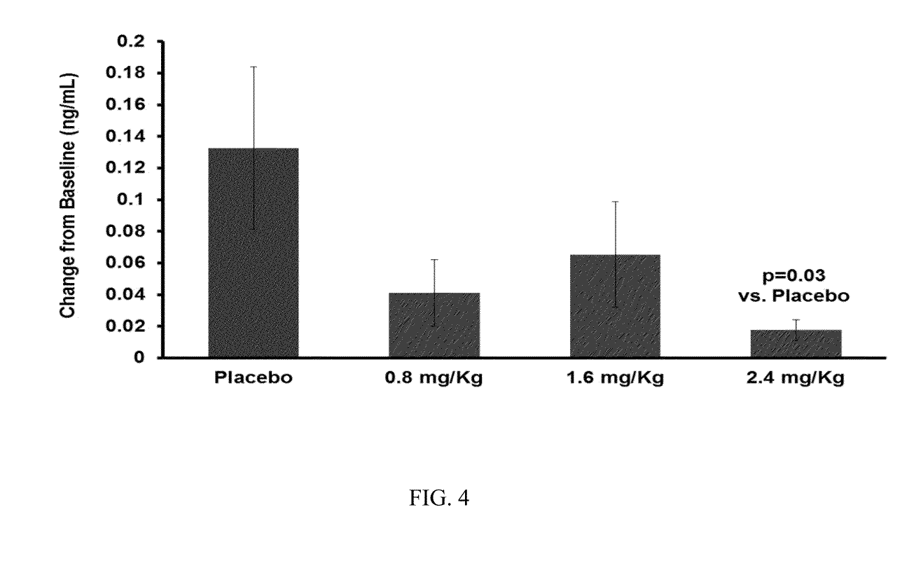

The change from baseline of TnT values at 24 hours was highest in the placebo group (0.132 ng/mL) and lowest in the 2.4 mg/kg dose group (0.018 ng/mL). Since the positive mean change from baseline in TnT values at 24 hours is indicative of myocardial injury, these data indicate that the 2.4 mg/kg dose group had the least myocardial injury. Between the placebo and 2.4 mg/kg dose groups, the changes of base line value of Troponin-T at 24 hours were statistically significant (p=0.0285). These data are shown in Table 7 and FIG. 4.

TABLE 7
Summary of Evaluation Parameters - Troponin-T (ng/mL) -
Change from baseline (FAP population).

| | | Treatment | | | | |
|---|---|---|---|---|---|---|
| | | | (R)Lip-EA-OH | | | Between |
| | | Placebo | 0.8 mg/kg | 1.6 mg/kg | 2.4 mg/kg | Groups |
| Day | Statistic | (N = 35) | (N = 36) | (N = 35) | (N = 36) | p-value (2] |
| Day 1 | n | 31 | 33 | 29 | 32 | |
| (24 hours) | Mean | 0.132 | 0.041 | 0.068 | 0.018 | 0.0747 |
| | Std Dev | 0.287 | 0.121 | 0.186 | 0.037 | |
| | Median | 0.000 | 0.000 | 0.000 | 0.000 | |
| | Range | −0.010-1.160 | 0.000-0.670 | 0.000-0.940 | 0.000-0.150 | |
| | Within Group | 0.0155 | 0.0595 | 0.0599 | 0.0121 | |
| | p-Value (1] | | | | | |
| | Between Group | | | | 0.0285 | |
| | p-Value (3] | | | | | |

N = Number of subjects with Troponin-T at the given time point. Baseline is the value from the Screening visit. At Baseline most of the measurements were below the level of detection (which is 0.001) hence statistics are shown with three digits.
(1] p-value from a paired t-test.
(2] p-value from an ANOVA model, Dunnett's test comparing the active treatment groups with placebo as control.
(3] Between groups p-value for placebo and (R)Lip-EA-OH 2.4 mg/kg groups based on t-test for means.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An aqueous pharmaceutical formulation, comprising:
(i) a compound represented by the following structural formula: 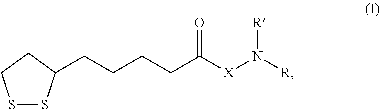
wherein:
R is (C₁-C₁₈)alkyl, (C₆-C₁₈)aryl or (C₆-C₁₈)aryl(C₁-C₁₈)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆-C₁₈)aryl or (C₆-C₁₈)aryl(C₁-C₁₈)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl;
R′ is hydrogen or (C₁-C₁₈)alkyl, wherein (C₁-C₁₈)alkyl is optionally substituted with one or more acidic substituents selected from the group consisting of —CO₂H, —SO₃H, —PO₃H₂, —OSO₃H, —OPO₃H₂, —B(OH)₂ and —NHOH; and
X is absent or is an amino acid, wherein the amino acid is oriented to form an amide linkage with 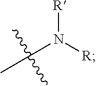
and
(ii) an inorganic base in an amount sufficient to deprotonate each acidic substituent in the compound of Structural Formula I,
wherein the formulation has a pH of from about 6.5 to about 8.0 and a tonicity of from about 250 mOsm to about 350 mOsm.

2. The aqueous pharmaceutical formulation of claim 1, wherein R is (C₁-C₃)alkyl and is substituted with at least one acidic substituent selected from the group consisting of —CO₂H, —SO₃H, —PO₃H₂, —OSO₃H, —OPO₃H₂, —B(OH)₂ and —NHOH.

3. The aqueous pharmaceutical formulation of claim 1, wherein R is (C₆)aryl and is substituted with at least one acidic substituent selected from the group consisting of —CO₂H, —SO₃H, —PO₃H₂, —OSO₃H, —OPO₃H₂, —B(OH)₂ and —NHOH, and is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

4. The aqueous pharmaceutical formulation of claim 1, wherein R is (C₆)aryl(C₁-C₃)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆)aryl(C₁-C₃)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl.

5. The aqueous pharmaceutical formulation of claim 1, wherein X is an amino acid and R′ is hydrogen.

6. The aqueous pharmaceutical formulation of claim 5, wherein X is aspartic acid, tyrosine, glutamic acid or alanine.

7. The aqueous pharmaceutical formulation of claim 2, wherein R is (C₁-C₃)alkyl substituted with one or two acidic substituents each independently selected from the group consisting of —CO₂H, —SO₃H, —PO₃H₂, —OSO₃H and —OPO₃H₂.

8. The aqueous pharmaceutical formulation of claim 1, wherein X is absent and R′ is hydrogen.

9. The aqueous pharmaceutical formulation of claim 8, wherein R is (C₁-C₃)alkyl substituted with one or two acidic substituents each independently selected from the group consisting of —CO₂H, —SO₃H, —PO₃H₂, —OSO₃H and —OPO₃H₂.

10. The aqueous pharmaceutical formulation of claim 8, wherein R is (C₆)aryl(C₁-C₃)alkyl substituted with one or two acidic substituents each independently selected from —CO₂H, —SO₃H, —PO₃H₂, —OSO₃H and —OPO₃H₂, and wherein aryl is optionally substituted with halo or hydroxy.

11. The aqueous pharmaceutical formulation of claim 8, wherein R is (C₂)alkyl substituted with one or two acidic substituents each independently selected from —CO₂H, —SO₃H, —PO₃H₂, —OSO₃H and —OPO₃H₂.

12. The aqueous pharmaceutical formulation of claim 8, wherein R is (C₆)aryl substituted with one acidic substituent selected from the group consisting of —CO₂H, —SO₃H, —PO₃H₂, —OSO₃H and —OPO₃H₂.

13. The aqueous pharmaceutical formulation of claim 1, wherein R′ is (C₁-C₃)alkyl.

14. The compound of claim 1, wherein X is absent and R and R′ are each (C₁-C₃)alkyl substituted with one acidic substituent selected from the group consisting of —CO₂H, —SO₃H, —PO₃H₂, —OSO₃H and —OPO₃H₂.

15. The aqueous pharmaceutical formulation of claim 1, wherein the compound is represented by the following structural formula:

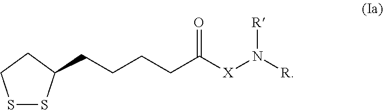

16. The aqueous pharmaceutical formulation of claim 1, wherein the compound is represented by the following structural formula:

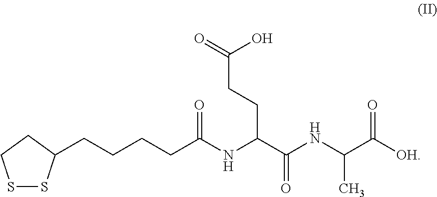 [SMILES: CC(NC(=O)C(CCC(=O)O)NC(=O)CCCCC1CCSS1)C(=O)O]

17. The aqueous pharmaceutical formulation of claim 1, wherein the compound is represented by the following structural formula:

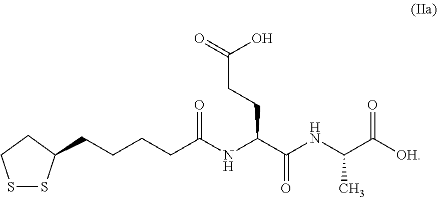 [SMILES: C[C@H](NC(=O)[C@H](CCC(=O)O)NC(=O)CCCC[C@@H]1CCSS1)C(=O)O]

18. The aqueous pharmaceutical formulation of claim 1, wherein the compound of Structural Formula I is represented by any one of Compounds A-AI in Table A.

19. The aqueous pharmaceutical formulation of claim 1, wherein the compound of Structural Formula I is represented by any one of Compounds A′-AI′ in Table A.

20. The aqueous pharmaceutical formulation of claim 1, wherein the formulation has a pH of from about 6.8 to about 7.6.

21. The aqueous pharmaceutical formulation of claim 20, wherein the formulation has a pH of from about 7.0 to about 7.2.

22. The aqueous pharmaceutical formulation of claim 1, wherein the formulation has a tonicity of from about 260 mOsm to about 320 mOsm.

23. The aqueous pharmaceutical formulation of claim 1, further comprising a buffer.

24. The aqueous pharmaceutical formulation of claim 23, wherein the buffer is phosphate buffer.

25. The aqueous pharmaceutical formulation of claim 1, further comprising a tonicity agent.

26. The aqueous pharmaceutical formulation of claim 25, wherein the tonicity agent is an ionic tonicity agent.

27. The aqueous pharmaceutical formulation of claim 26, wherein the ionic tonicity agent is sodium chloride.

28. The aqueous pharmaceutical formulation of claim 1, wherein the formulation comprises from about 5 mg/mL to about 50 mg/mL of the compound of Structural Formula I.

29. The aqueous pharmaceutical formulation of claim 28, wherein the formulation comprises from about 9 mg/mL to about 30 mg/mL of the compound of Structural Formula I.

30. The aqueous pharmaceutical formulation of claim 29, wherein the formulation comprises about 25 mg/mL of the compound of Structural Formula I.

31. The aqueous pharmaceutical formulation of claim 1, wherein the inorganic base is a sodium base.

32. The aqueous pharmaceutical formulation of claim 1, wherein the inorganic base is a hydroxide base.

33. The aqueous pharmaceutical formulation of claim 1, wherein the inorganic base is sodium hydroxide.

34. The aqueous pharmaceutical formulation of claim 1, wherein the formulation comprises from about 25 mg/mL to about 200 mg/mL inorganic base.

35. The aqueous pharmaceutical formulation of claim 34, wherein the formulation comprises from about 50 mg/mL to about 150 mg/mL inorganic base.

36. The aqueous pharmaceutical formulation of claim 1, comprising:
(i) from about 5 mg/mL to about 50 mg/mL of the compound of Structural Formula I; and
(ii) from about 25 mg/mL to about 200 mg/mL sodium hydroxide;
wherein the formulation has a pH of from about 7.0 to about 7.2 and a tonicity of from about 260 mOsm to about 320 mOsm.

37. The aqueous pharmaceutical formulation of claim 36, comprising:
from about 9 mg/mL to about 30 mg/mL of the compound of Structural Formula I; and
(ii) from about 50 mg/mL to about 150 mg/mL sodium hydroxide;
wherein the formulation has a pH of from about 7.0 to about 7.2 and a tonicity of from about 260 mOsm to about 320 mOsm.

38. The aqueous pharmaceutical formulation of claim 1, wherein the formulation is substantially free of polymerized compound of Structural Formula I.

39. A process for preparing an aqueous pharmaceutical formulation, comprising:
a) providing a compound represented by the following structural formula: 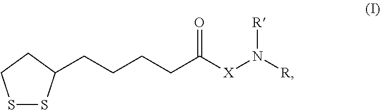
wherein:
R is (C₁-C₁₈)alkyl, (C₆-C₁₈)aryl or (C₆-C₁₈)aryl(C₁-C₁₈)alkyl and is substituted with at least one acidic substituent selected from the group consisting of —CO₂H, —SO₃H, —PO₃H₂, —OSO₃H, —OPO₃H₂, —B(OH)₂ and —NHOH, wherein the aryl of the (C₆-C₁₈)aryl or (C₆-C₁₈)aryl(C₁-C₁₈)alkyl is optionally further substituted with one or more substituents selected from the group consisting of hydroxy, halo, (C₁-C₃)alkyl, halo(C₁-C₃)alkyl, cyano, nitro, (C₁-C₃)alkoxy and thio(C₁-C₃)alkyl;
R′ is hydrogen or (C₁-C₁₈)alkyl, wherein (C₁-C₁₈)alkyl is optionally substituted with one or more acidic substituents selected from the group consisting of —CO₂H, —SO₃H, —PO₃H₂, —OSO₃H, —OPO₃H₂, —B(OH)₂ and —NHOH; and
X is absent or is an amino acid, wherein the amino acid is oriented to form an amide linkage with 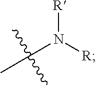
b) providing an aqueous solution comprising an inorganic base in an amount sufficient to deprotonate each acidic substituent in the compound of Structural Formula I, wherein the volume of the aqueous solution is equal to or greater than about 75% of the volume of the formulation;
c) adding the compound of Structural Formula I to the aqueous solution, thereby forming a pharmaceutical solution; and
d) diluting the pharmaceutical solution to the volume of the formulation with a diluent,
thereby preparing the aqueous pharmaceutical formulation.

40. A process for preparing an aqueous pharmaceutical formulation having a pH of from about 6.5 to about 8.0 and a tonicity of from about 250 mOsm to about 350 mOsm, comprising:
a) providing an aqueous solution comprising a buffer, a tonicity agent and sodium hydroxide, wherein the amount of sodium hydroxide in the aqueous solution is an amount sufficient to form a formulation comprising from about 25 mg/mL to about 200 mg/mL sodium hydroxide, and the volume of the aqueous solution is equal to or greater than about 75% of the volume of the formulation;
b) adding a compound represented by the following structural formula: 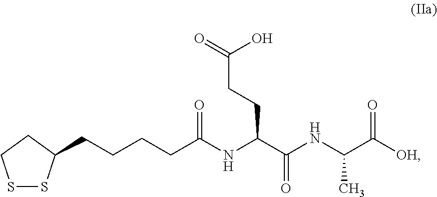 [SMILES: C[C@H](NC(=O)[C@H](CCC(=O)O)NC(=O)CCCC[C@@H]1CCSS1)C(=O)O]
to the aqueous solution in an amount sufficient to form a formulation comprising from about 5 mg/mL to about 50 mg/mL of the compound, thereby forming a pharmaceutical solution; and
c) diluting the pharmaceutical solution to the volume of the formulation with a diluent,
thereby preparing the aqueous pharmaceutical formulation having a pH of from about 6.5 to about 8.0 and a tonicity of from about 250 mOsm to about 350 mOsm.

41. The aqueous pharmaceutical formulation of claim 40, wherein the compound of Structural Formula IIa is added in an amount sufficient to form a formulation comprising about 25 mg/mL of the compound represented by Structural Formula IIa.

42. The process of claim 40, comprising:
a) providing an aqueous solution comprising sodium phosphate dibasic, sodium chloride and sodium hydroxide, wherein the amount of sodium hydroxide in the aqueous solution is an amount sufficient to form a formulation comprising about 125 mg/mL sodium hydroxide, and the volume of the aqueous solution is equal to or greater than about 75% of the volume of the formulation;
b) adding a compound represented by the following structural formula: 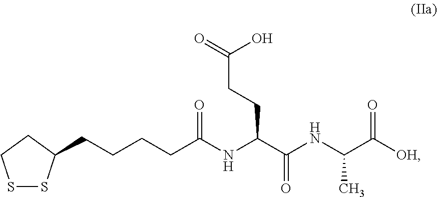 [SMILES: C[C@H](NC(=O)[C@H](CCC(=O)O)NC(=O)CCCC[C@@H]1CCSS1)C(=O)O]
to the aqueous solution in an amount sufficient to form a formulation comprising about 25 mg/mL of the compound, thereby forming a pharmaceutical solution; and
c) diluting the pharmaceutical solution to the volume of the formulation with a diluent,
thereby preparing the aqueous pharmaceutical formulation having a pH of from about 7.0 to about 7.2 and a tonicity of from about 260 mOsm to about 320 mOsm.

43. The process of claim 40, wherein the formulation is substantially free of polymerized compound of Structural Formula I.

44. The process of claim 40, further comprising adjusting the pH of the pharmaceutical solution.

45. The process of claim 40, wherein the volume of the aqueous solution is equal to or greater than about 90% of the volume of the formulation.

46. The process of claim 40, wherein the diluent is water.

47. The process of claim 40, wherein the aqueous pharmaceutical formulation has a pH of from about 6.5 to about 8.0 and a tonicity of from about 250 mOsm to about 350 mOsm.

48. An aqueous pharmaceutical formulation made according to the process of any one of claim 39.

49. An aqueous pharmaceutical formulation made according to the process of claim 40.
